(19) United States
(12) Patent Application Publication
(10) Pub. No.: US 2022/0273719 A1
(43) Pub. Date: Sep. 1, 2022

(54) GENETICALLY MODIFIED NK CELLS AND USES THEREOF

(71) Applicant: NATIONAL UNIVERSITY OF SINGAPORE, Singapore (SG)

(72) Inventors: Shu WANG, Singapore (SG); Yu Yang NG, Singapore (SG)

(21) Appl. No.: 17/632,877

(22) PCT Filed: Aug. 11, 2020
(86) PCT No.: PCT/SG2020/050463

§ 371 Date: Feb. 4, 2022

(30) Foreign Application Priority Data
Aug. 8, 2019 (SG) 10201907378Q

(51) Int. Cl. A61K 35/17; C12N 5/0783; C12N 15/62; C12N 15/86; C07K 14/715; C07K 14/725; C07K 14/705; C07K 14/47; C07K 16/30; A61P 35/00

(52) U.S. Cl. CPC A61K 35/17; C12N 5/0646; C12N 15/625; C12N 15/86; C07K 14/715; C07K 14/7051; C07K 14/70517; C07K 14/705; C07K 14/4748; C07K 14/7056; C07K 16/30; A61P 35/00; C12N 2501/2302; C12N 2502/99; C12N 2710/14043; C07K 2319/02; C07K 2319/03; C07K 2319/10; C07K 2317/622; C07K 2317/569; C07K 2319/33; C07K 2319/30; A61K 2039/5156

(57) ABSTRACT

Disclosed herein include a natural killer (NK) cell genetically modified to comprise a recombinant nucleic acid encoding C-X-C Motif Chemokine Receptor 1 (CXCR1), a pharmaceutical composition comprising the NK cell, methods of preparing the NK cell, and method of treating cancer or tumor using the NK cell.

### CROSS-REFERENCE TO RELATED APPLICATIONS

This application claims the benefit of priority of the Singapore application No. 10201907378Q, filed on 8 Aug. 2019, the contents of it being hereby incorporated by reference in its entirety for all purposes.

### FIELD OF THE INVENTION

The present invention relates generally to the field of biotechnology and cell therapy. In particular, the present invention relates to genetically modified NK cells for cancer immunotherapy, their preparation method and use in patients in need thereof.

### BACKGROUND OF THE INVENTION

Recent years have seen tremendous progress in utilizing immune effector cells for cancer therapy. Cancer immunotherapy employing genetically engineered T lymphocytes that express chimeric antigen receptors (CARs) is an effective approach in treating several haematological cancers. CARs are composed of an extracellular antigen-binding domain, typically the single-chain variable fragment (scFv) derived from a monoclonal antibody, and an intracellular signaling domain. Despite recent success in clinical trials, several major limitations are associated with CAR-modified T cells. In addition, the need to generate an autologous CAR-T cell product for each individual patient is logistically demanding and restrictive for a wider adoption in the medical practice. Secondly, manufacturing patient specific CAR-T cells often takes several weeks, which is a significant drawback for treating patients with advancing disease. Furthermore, it is not always possible to collect enough lymphocytes from heavily pretreated patients to generate sufficient quantities of CAR-T cells. An allogeneic “off-the-shelf” product could overcome these challenges, but allogeneic T cells pose significant risk of graft-versus-host disease (GVHD).

Natural killer cells (NK cells) are a type of lymphocyte that is a part of the innate immune system. NK cells play an important role in cancer immune-surveillance. In contrast to T cells, they do not require prior sensitization and recognition of peptide antigens presented in complex with major histocompatibility complex (MHC) molecules. Instead, NK cell cytotoxicity can be triggered rapidly upon appropriate stimulation through an array of native receptors, which in principle could reduce the risk of relapse or resistance mediated by loss of CAR-targeted antigen. Importantly, a favorable safety profile without obvious toxicity following the transfer of allogeneic NK cells in clinical settings (lack of the potential to cause GVHD) has been considered as an important practical advantage over T cell therapy. Therefore, adoptive cell transfer therapy with natural killer (NK) cells is one of the most promising immunotherapeutic modalities for cancer patients. However, the success of the approach has so far been limited to patients with haematological malignancies. Furthermore, the translation of NK cell therapy to non-haematological malignancies is challenging due to the special pathophysiological characteristics of solid tumors, including target antigen heterogeneity, obstacles to CAR immune cell trafficking, and intrinsic negative regulatory mechanisms of tumor microenvironment.

Therefore, it is an objective of the present invention to provide improved NK cells for cancer immunotherapy, which address some or all of the above mentioned problems.

### SUMMARY OF THE INVENTION

In one aspect, the present invention discloses a natural killer (NK) cell genetically modified to comprise a recombinant nucleic acid encoding C-X-C Motif Chemokine Receptor 1 (CXCR1).

In another aspect, the present invention discloses a pharmaceutical composition comprising a pharmaceutically effective amount of the NK cell of the present invention and a pharmaceutically acceptable excipient.

In another aspect, the present invention discloses a method of treating cancer or tumor in a subject in need thereof, comprising administering to the subject a pharmaceutically effective amount of the NK cell of the present invention, or the pharmaceutical composition of the present invention.

In another aspect, the present invention discloses a method of treating cancer or tumor in a subject in need thereof, the method comprises: (i) obtaining NK cells from the subject, or from a donor which is different from the subject to be treated; (ii) providing a recombinant nucleic acid encoding CXCR1; (iii) transferring the recombinant nucleic acid encoding CXCR1 into the NK cell to obtain genetically modified NK cells; and (iv) administering to the subject a pharmaceutically effective amount of the NK cells obtained from (iii).

In another aspect, the present invention discloses a method of treating cancer or tumor in a subject in need thereof, the method comprises: (i) obtaining NK cells from the subject, or from a donor which is different from the subject to be treated; (ii) providing a recombinant nucleic acid encoding CXCR1 and a recombinant nucleic acid encoding a recombinant chimeric antigen receptor (CAR); (iii) transferring the recombinant nucleic acid encoding CXCR1 and the recombinant nucleic acid encoding the recombinant CAR into the NK cell to obtain genetically modified NK cells; and (iv) administering to the subject a pharmaceutically effective amount of the NK cells obtained from (iii).

In another aspect, the present invention discloses a method of preparing the NK cell of the present invention, the method comprises: (i) obtaining or providing NK cells; (ii) providing a recombinant nucleic acid encoding CXCR1; and (iii) transferring the recombinant nucleic acid encoding CXCR1 into the NK cell.

In a further aspect, the present invention discloses a method of preparing the NK cell of the present invention, the method comprises: (i) obtaining or providing NK cells; (ii) providing a recombinant nucleic acid encoding CXCR1 and a recombinant nucleic acid encoding the recombinant CAR; and (iii) transferring the recombinant nucleic acid encoding CXCR1 and the recombinant nucleic acid encoding the recombinant CAR into the NK cell.

### BRIEF DESCRIPTION OF THE DRAWINGS

The invention will be better understood with reference to the detailed description when considered in conjunction with the non-limiting examples and the accompanying drawings, in which:

FIG. 1 is a schematic overview of the design concepts for constructs useful for modifying the NK cells as described in the present disclosure (A-C). The particular constructs as described in the examples of this disclosure (D, E) are used for generating recombinant mRNA transcripts of CXCR1 (D) and NKG2D chimeric antigen receptor (CAR) (E).

FIG. 2 are flow cytometry histograms showing the expression of CXCR1 in NK cells. (A) CXCR1 expression was seen in most freshly isolated NK cells. (B) CXCR1 expression was almost completely lost in NK cells after ex vivo expansion as outlined in the Methods section, with an expression profile almost identical to that of the isotype control. (C) The large majority of the ex vivo expanded NK cells expressed CXCR1 after electroporation with CXCR1 mRNA, while the ex vivo expanded NK cells which were electroporated without CXCR1 mRNA (‘mock’) showed little or no expression of CXCR1. The results show that electroporation of NK cells with mRNA encoding CXCR1 successfully restored and elevated CXCR1 expression in the ex vivo expanded NK cells.

FIG. 3 is a histogram showing the migration of CXCR1 transfected NK cells towards three different human cancer cells. In vitro migration of NK cells electroporated with CXCR1 mRNA towards conditioned media derived from different ovarian cancer cell lines (SKOV3, CaOV3 and SW626) were assessed and compared with that of NK cells electroporated without the addition of any mRNA (mock NK cells). The ability to migrate towards IL-8 secreting tumor cells is better in NK cells electroporated with CXCR1 mRNA compared to mock NK cells. The results show that CXCR1 overexpression increases the migration ability of ex vivo expanded NK cells towards cancer cells.

FIG. 4 shows the results of the cell migration studies of CXCR1 overexpressing NK cells and mock NK cells towards SKOV3 intraperitoneal xenografts. The SKOV3 xenograft model was established by intraperitoneal injection of SKOV3-luc cells. Seven days after tumor inoculation, mice were imaged using luciferin to confirm tumor presence in the peritoneum. Mice were then assigned based on similar tumor burden prior to the intravenous tail vein injection of DiR-labelled NK cells. (A) Bioluminescence images to show tumor growth in the two groups of mice. (B) In vivo migration of mock NK cells versus CXCR1 overexpressing NK cells towards SKOV3 xenografts. After NK cell injection, mice were imaged 24 and 48 hours later. (C) Ex vivo imaging of isolated tumors from the peritoneum 48 hours after NK cell injection. Tumors obtained from mice without NK cell treatment served as controls (control tumor). Flux values of the isolated tumors are shown on the right. ****: P<0.0001 statistical significance between CXCR1 overexpressing NK cells and mock NK cells. The results show that overexpression of CXCR1 in NK cells can improve the migration of intravenously (i.v.) injected NK cells towards peritoneal tumors.

FIG. 5 shows the results of the studies of in vivo killing capacities of CXCR1 overexpressing NK cells and mock NK cells against intraperitoneal (i.p) xenografts of human SKOV3 ovarian cancer cells. SKOV3-luc cells were i.p injected to establish a peritoneal ovarian carcinoma xenograft model. Seven days after inoculation, tumor burden was confirmed by luciferin imaging and NK cells were injected as outlined in Material and Methods. (A) Bioluminescence images at day 7 and day 21 post-tumor inoculation are shown. (B) Flux values of tumor burden at day 7 and day 21 were plotted in the bar graph. *: P<0.05 statistical significance between CXCR1 overexpressing NK cells and mock NK cells at day 21. The results show that CXCR1 overexpressing NK cells display higher killing capacity against cancer cells, as compared to mock NK cells.

FIG. 6 shows the results of the studies of in vivo killing capacities of NKG2D CAR-modified, CXCR1 overexpressing NK cells and NKG2D CAR-modified NK cells without CXCR1 overexpression against tumor cells. The SKOV3 xenograft model was established by intraperitoneal injection of SKOV3-luc cells. Seven days after tumor inoculation, mice were imaged using luciferin to confirm tumor presence in the peritoneum. Mice were then assigned based on similar tumor burden prior to the intravenous tail vein injection of NK cells. NK cells were injected as outlined in the Method section. (A) Bioluminescence images at day 7 and day 21 post-tumor inoculation are shown. (B) Flux values of tumor burden at day 7 and day 21 were plotted in the bar graph. **: P<0.01 statistical significance between NKG2D CAR-modified, CXCR1 overexpressing NK cells and NKG2D CAR-modified NK cells without CXCR1 at day 21. The results show that killing capacity of NKG2D CAR-modified, CXCR1 overexpressing NK cells against tumor cells is higher than that of NKG2D CAR-modified NK cells without CXCR1 overexpression.

FIG. 7 shows the results of the studies of in vitro cancer cell killing capacities of EpCAM CAR-modified, CXCR1 overexpressing NK cells. NK cells were electroporated with an mRNA encoding anti-EpCAM CAR. The results show that modified NK cells showed an improved in vitro killing capacity against SKOV3 cancer cells as compared to mock-control NK cells, demonstrating the potential of CXCR1 modified CAR NK cells to kill tumor cells expressing targets other than NKG2D ligands. Therefore, a scFv of a monoclonal antibody (in this case an anti-EPCAM) can also be used in the recombinant CAR, which together with overexpressed CXCR1 in NK cells leads to improved killing capacity against ovarian cancer cells.

FIG. 8 shows the results of studies of in vivo tumor infiltration of CXCR1 overexpressing NK cells in a FaDu subcutaneous xenograft model. Mice were inoculated with FaDu human hypopharyngeal cancer cells subcutaneously at the left flank, and 10 days later DiR-labelled NK cells were intravenously injected through the tail vein. After the NK cell injection, mice were imaged every 24 hours up till 72 hours. (A) Whole-body imaging of mice. Flux values at the tumour sites (circled) were plotted in the bar graph, n=5. (B) Ex vivo imaging of isolated tumours from the right flank 72 hours after NK cell injection. Tumours obtained from mice without NK cell injection served as control. Flux values of the isolated tumours are shown on the right, n=5. *: P<0.05, statistical significance between CXCR1 overspressing NK cells and mock NK cells. The results show that overexpression of CXCR1 in NK cells can improve the migration/infiltration of i.v.-injected NK cells toward subcutaneous tumors.

### DEFINITIONS

A “genetically modified cell” means any cell of any organism that is modified, transformed, or manipulated by addition or modification of a gene, a DNA or RNA molecule, or protein or polypeptide.

The term “natural killer cell” or “NK cell” as used herein refers to a type of cytotoxic lymphocyte critical to the innate immune system. NK cells are large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter into the circulation. NK cells provide rapid responses to virus-infected cells, and respond to tumor formation. Typically, immune cells detect major histocompatibility complex (MHC) presented on infected cell surfaces, triggering cytokine release, causing lysis or apoptosis. NK cells are different, however, as they have the ability to recognize stressed cells in the absence of antibodies and MHC, allowing for a much faster immune reaction. They were named “natural killers” because of the initial notion that they do not require activation to kill cells that are missing markers of MHC class I. This role is especially important because harmful cells that are missing MHC I markers cannot be detected and destroyed by other immune cells, such as T lymphocyte cells. Since NK cell action is non-MHC-restricted, it is not necessary to match the histocompatibility complex of the individual patient in use, that is, NK cells can be used for cell therapy of allogeneic patients, and have wide clinical application value.

The term “C-X-C Motif Chemokine Receptor 1 (CXCR1)” as used herein refers to interleukin 8 receptor alpha, which is a chemokine receptor. In human, it is encoded by the gene CXCR1 (GeneCards identifier: GCO2M218162). CXCR1 is a member of the G-protein-coupled receptor family. It is a receptor for interleukin 8 (IL8), which binds to IL8 with high affinity. CXCR1 also binds to C-X-C Motif Ligand 6 (CXCL6) with high affinity. In one specific example, the nucleotide sequence encoding human CXCR1 is 5′-ATGTCAAATATTACAGATCCACAGATGTGGGATTTTGATGATCTAAATTTCACTG GCATGCCACCTGCAGATGAAGATTACAGCCCCTGTATGCTAGAAACTGAGACAC TCAACAAGTATGTTGTGATCATCGCCTATGCCCTAGTGTTCCTGCTGAGCCTGCT GGGAAACTCCCTGGTGATGCTGGTCATCTTATACAGCAGGGTCGGCCGCTCCGTC ACTGATGTCTACCTGCTGAACCTGGCCTTGGCCGACCTACTCTTTGCCCTGACCTT GCCCATCTGGGCCGCCTCCAAGGTGAATGGCTGGATTTTTGGCACATTCCTGTGC AAGGTGGTCTCACTCCTGAAGGAAGTCAACTTCTACAGTGGCATCCTGCTGTTGG CCTGCATCAGTGTGGACCGTTACCTGGCCATTGTCCATGCCACACGCACACTGAC CCAGAAGCGTCACTTGGTCAAGTTTGTTTGTCTTGGCTGCTGGGGACTGTCTATG AATCTGTCCCTGCCCTTCTTCCTTTTCCGCCAGGCTTACCATCCAAACAATTCCAG TCCAGTTTGCTATGAGGTCCTGGGAAATGACACAGCAAAATGGCGGATGGTGTT GCGGATCCTGCCTCACACCTTTGGCTTCATCGTGCCGCTGTTTGTCATGCTGTTCT GCTATGGATTCACCCTGCGTACACTGTTTAAGGCCCACATGGGGCAGAAGCACC GAGCCATGAGGGTCATCTTTGCTGTCGTCCTCATCTTCCTGCTTTGCTGGCTGCCC TACAACCTGGTCCTGCTGGCAGACACCCTCATGAGGACCCAGGTGATCCAGGAG AGCTGTGAGCGCCGCAACAACATCGGCCGGGCCCTGGATGCCACTGAGATTCTG GGATTTCTCCATAGCTGCCTCAACCCCATCATCTACGCCTTCATCGGCCAAAATTT TCGCCATGGATTCCTCAAGATCCTGGCTATGCATGGCCTGGTCAGCAAGGAGTTC TTGGCACGTCATCGTGTTACCTCCTACACTTCTTCGTCTGTCAATGTCTCTTCCAA CCTCTGA-3′ (SEQ ID NO: 1) (NCBI Reference Sequence: NM_000634.3). In this specific example, the amino acid sequence of the CXCR1 is (from the N-terminal to the C-terminal):

(SEQ ID NO: 2)
MSNITDPQMWDFDDLNFTGMPPADEDYSPCMLETETLNKYVVIIAYALVF
LLSLLGNSLVMLVILYSRVGRSVTDVYLLNLALADLLFALTLPIWAASKV
NGWIFGTFLCKVVSLLKEVNFYSGILLLACISVDRYLAIVHATRTLTQKR
HLVKFVCLGCWGLSMNLSLPFFLFRQAYHPNNSSPVCYEVLGNDTAKWRM
VLRILPHTFGFIVPLFVMLFCYGFTLRTLFKAHMGQKHRAMRVIFAVVLI
FLLCWLPYNLVLLADTLMRTQVIQESCERRNNIGRALDATEILGFLHSCL
NPIIYAFIGQNFRHGFLKILAMHGLVSKEFLARHRVTSYTSSSVNVSSN
L.

The term “chimeric antigen receptor” or the short form “CAR” as used herein refers to artificial receptor proteins, or chimeric immunoreceptors, and encompass engineered receptors that graft an artificial specificity onto a particular immune effector cell. CARs may be employed to impart the specificity of a monoclonal antibody onto a NK cell, thereby allowing a large number of specific NK cells to be generated, for example, for use in adoptive cell therapy. CARs typically comprise an intracellular activation domain, a transmembrane domain, and an extracellular domain comprising an antigen binding region. CARs can combine antibody-based specificity for a desired antigen with a NK cell receptor-activating intracellular domain to generate a chimeric protein that exhibits a specific cellular immune activity such as anti-tumor cellular immune activity. In some cases, molecules can be co-expressed with the CAR, including co-stimulatory molecules, reporter genes for imaging, gene products that conditionally ablate the NK cells upon addition of a pro-drug, homing receptors, chemokines, chemokine receptors, cytokines, and cytokine receptors.

As used herein, the term “antigen” is a molecule capable of being bound by an antibody or cell surface receptor. An antigen may generally be used to induce a humoral immune response and/or a cellular immune response leading to the production of lymphocytes.

As used herein, the term “antibody” refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. An “antibody” includes monoclonal, polyclonal, bispecific, multispecific, murine, chimeric, humanized and human antibodies.

As used herein, the term “antibody fragment” is a portion of an intact antibody such as F(ab′) 2, F(ab) 2, Fab′, Fab, Fv, scFv, or dAb. Regardless of structure, an antibody fragment as used herein binds with the same antigen that is recognized by the full-length antibody. For example, antibody fragments include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). “Single-chain antibodies”, often abbreviated as “scFv” consist of a polypeptide chain that comprises both a VH and a VL domain which interact to form an antigen-binding site. The VH and VL domains are usually linked by a peptide of 1 to 25 amino acid residues. Antibody fragments also include diabodies, triabodies and single domain antibodies (dAb). In some examples, a “fragment” of an antibody comprises an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of the antibody.

“Specifically” or “selectively” binds, when referring to a ligand/receptor, nucleic acid/complementary nucleic acid, antibody/antigen, or other binding pair (e.g., a cytokine to a cytokine receptor) indicates a binding reaction which is determinative of the presence of the protein in a heterogeneous population of proteins and other biologies. Thus, under designated conditions, a specified ligand binds to a particular receptor and does not bind in a significant amount to other proteins present in the sample. Specific binding can also mean, e.g., that the binding compound, nucleic acid ligand, antibody, or binding composition derived from the antigen-binding site of an antibody, binds to its target with an affinity that is at least 25% greater, at least 50% greater, at least 100% (2-fold) greater, at least five times greater, at least ten times greater, at least 20-times greater, or at least 100-times greater than the affinity with any other binding compound.

Natural-killer group 2, member D, also known as Klrk1 (NKG2D), is a C-type lectin-like receptor, which was firstly identified in NK cells as an activating immune receptor. NKG2D is a type II transmembrane glycoprotein, which does not contain any known signaling elements in the intracellular domain. Resembling many activating receptors, NKG2D depends on an adaptor molecule to initiate signaling transduction and cellular activation. In human, NKG2D is not only expressed by all NK cells, but is also expressed by all CD8⁺ T cells, and subsets of γδ⁺ T cells as a co-stimulatory receptor. NKG2D expression and signaling can be regulated by cytokines and tumor-derived factors. Cytokines, such as IL-2, IL-7, IL-12, IL-15, and type I interferons (IFNs) increase cell surface expression of NKG2D. Cytokines such as IL-21, IFN-γ, and TGF-β have been shown to decrease NKG2D expression. IL-21 has been reported to reduce expressions of NKG2D in human CD8⁺ T cells and NK cells. In mice, IL-21 stimulation of NK cells is dependent on regulating the NKG2D expression in mouse model of breast carcinoma.

Epithelial cell adhesion molecule (EpCAM) is a transmembrane glycoprotein mediating Ca²⁺-independent homotypic cell-cell adhesion in epithelia. EpCAM in normal epithelia is expressed mostly on the basolateral membrane, and is therefore expected to be much less accessible to antibodies than EpCAM in cancer tissue, where it is located on the surface of the cancer cells. The terms “polynucleotide”, “nucleic acid” and “oligonucleotide” are used interchangeably and refer to a polymeric form of nucleotides of any length, either deoxyribonucleotides or ribonucleotides or analogues thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogues. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labelling component. The term also refers to both double- and single-stranded molecules. Unless otherwise specified or required, a polynucleotide encompasses both the double-stranded form and each of two complementary single-stranded forms known or predicted to make up the double-stranded form.

The term “recombinant nucleic acid” as used herein refers to nucleic acid formed by laboratory methods of genetic recombination (such as molecular cloning) to bring together genetic material from multiple sources. The nucleic acid sequences used in the construction of recombinant nucleic acid molecules can originate from any species. For example, human nucleic acid may be joined with bacterial nucleic acid. In addition, nucleic acid sequences that do not occur anywhere in nature may be created by the chemical synthesis of nucleic acid, and incorporated into recombinant molecules. Proteins that can result from the expression of recombinant nucleic acid within living cells are termed recombinant proteins. When recombinant nucleic acid encoding a protein is introduced into a host organism, the recombinant protein is not necessarily produced. Expression of foreign proteins requires the use of specialized expression vectors and often necessitates significant restructuring by foreign coding sequences.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

The term “operably linked” as used in the context of a promoter and a nucleic acid means that the promoter can be used to initiate transcription of that nucleic acid.

The terms “transfer” or “transfect” as used herein refer to the general process by which exogenous nucleic acid is introduced into the host cell, said process can be mechanical transfection (including electroporation), chemical transfection or viral transduction. A “transfected” cell is one which has been transfected with exogenous nucleic acid using any of the above mentioned methods. The cell includes the primary subject cell and its progeny.

As used herein, the term “autologous” is meant to refer to any material derived from the same individual to which it is later to be re-introduced into the individual.

As used herein, the term “allogeneic” refers to any material derived from an individual other than the individual to which it is later to be introduced into.

As used herein, “percent identity” refers to sequence identity between two peptides or between two nucleic acid molecules. Percent identity can be determined by comparing a position in each sequence which may be aligned for purposes of comparison. When a position in the compared sequence is occupied by the same base or amino acid, then the molecules are identical at that position.

As used herein, the terms “peptide”, “polypeptide”, and “protein” are used interchangeably, and refer to a compound having amino acid residues covalently linked by peptide bonds. A protein or peptide must contain at least two amino acids, and no limitation is placed on the maximum number of amino acids that can include a protein's or peptide's sequence. Polypeptides include any peptide or protein having two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the phrase “homologous” or “variant” nucleotide sequence, or “homologous” or “variant” amino acid sequence refers to sequences characterized by identity, at the nucleotide level or amino acid level, of at least a specified percentage. Homologous nucleotide sequences include those sequences coding for naturally occurring allelic variants and mutations of the nucleotide sequences set forth herein. Homologous nucleotide sequences include nucleotide sequences encoding for a protein of a mammalian species other than humans. Homologous amino acid sequences include those amino acid sequences which contain conservative amino acid substitutions and which polypeptides have the same binding and/or activity. In some examples, a homologous nucleotide or amino acid sequence has at least 60% or greater, for example at least 70%, or at least 75%, or at least 80%, or at least 85%, or at least 90%, or at least 95%, or at least 98%, or at least 99%, with a comparator sequence. In some examples, a homologous nucleotide or amino acid sequence has at least 90%, 91%, 92%, 93%, 94%, 95%, 96%, 97%, 98% or 99% identity with a comparator sequence. In some examples, a homologous amino acid sequence has no more than 15, or no more than 10, or no more than 5 or no more than 3 conservative amino acid substitutions. Percent identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman. In some examples, the recombinant nucleic acid molecules used to modify the NK cells according to the present disclosure are homologous to the exemplary nucleotide sequences disclosed herein, such as the sequences provided in any of SEQ ID NO: 1, 3, 4, 5, 6, 7, 9, 12, 13, 14, 15 or 16. In some other examples, the CXCR1 and/or chimeric antigen receptor (CAR) expressed in the modified NK cells are homologous to the exemplary amino acid sequences disclosed herein, such as the sequences provided in any of SEQ ID NO: 2, 8, 10 or 11.

The term “express” or “expression” refers to the production of a gene product in a cell. The term “overexpress” or “overexpression” refers to the artificial expression of a gene in increased quantity, as compared to the level of expression in a control cell. In some examples, the control cell is a primary NK cell isolated from a subject. In some examples, the control cell is an NK cell obtained from the expansion of a primary NK cell isolated from a subject. In some examples, the control cell is one of the ex vivo expanded NK cells from a subject, said cells have been transfected with an empty vector or without any nucleic acids.

The term “transient” when referred to expression means a polynucleotide is not incorporated into the genome of the cell. In contrast, the term “stable” when referred to expression means a polynucleotide is incorporated into the genome of the cell. Transient expression can occur from introduced constructs which contain expression signals functional in the host cell, but which constructs do not replicate and rarely integrate in the host cell, or where the host cell is not proliferating. Transient expression also can be accomplished by inducing the activity of a regulatable promoter operably linked to the gene of interest, although such inducible systems frequently exhibit a low basal level of expression. Stable expression can be achieved by introduction of a nucleic acid construct that can integrate into the host genome or that autonomously replicates in the host cell. Stable expression of the gene of interest can be selected for through the use of a selectable marker located on or transfected with the expression construct, followed by selection for cells expressing the marker. When stable expression results from integration, integration of constructs can occur randomly within the host genome or can be targeted through the use of constructs containing regions of homology with the host genome sufficient to target recombination with the host locus. Where constructs are targeted to an endogenous locus, all or some of the transcriptional and translational regulatory regions can be provided by the endogenous locus. To achieve expression in a host cell, the transformed nucleic acid is operably associated with transcriptional and translational initiation and termination regulatory regions that are functional in the host cell.

The term “tumor” as used herein refers to a swelling of a part of the body, generally without inflammation, caused by an abnormal growth of tissue. Tumors can be benign or malignant (i.e cancerous). A benign tumor does not invade nearby tissue or spread to other parts of the body. Common types of benign tumors include adenomas, fibromas (or fibroids), hemangiomas, lipomas, meningiomas, myomas, neuromas, and osteochondromas. Adenomas are benign tumors starting in the epithelial tissue of a gland or gland-like structure. A common type of adenoma is a polyp in the colon. Adenomas might also grow in the liver or the adrenal, pituitary, or thyroid gland. Fibromas (or fibroids) are tumors of fibrous or connective tissue that can grow in any organ. Hemangiomas are a buildup of blood vessel cells in the skin or internal organs. Lipomas grow from fat cells. They are the most common benign tumor in adults, often found in the neck, shoulders, back, or arms. Meningiomas are tumors that develop from the membrane surrounding the brain and spinal cord. Myomas are tumors that grow from muscle. Neuromas are tumors that develop from the nerves. Osteochondromas are tumors that develop from the bones.

The term “cancer” as used herein refers to an unregulated proliferation of cells due to loss or normal controls, resulting in unregulated growth, lack of differentiation, local tissue invasion, and, often, metastasis. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

### DETAILED DESCRIPTION OF THE PRESENT INVENTION

The inventors of the present application have found that after genetically modifying natural killer (NK) cells with a gene encoding C-X-C Motif Chemokine Receptor 1 (CXCR1) to induce the expression of CXCR1, the genetically modified NK cells display increased migration capacity towards tumor cell-released cytokines, as well as enhanced infiltration into solid tumors. The increased migration and enhanced infiltration resulted in increased antitumor responses of NK cells.

Thus, in one example, the present invention refers to a natural killer (NK) cell genetically modified to comprise a recombinant nucleic acid encoding C-X-C Motif Chemokine Receptor 1 (CXCR1). The introduction of the recombinant nucleic acid encoding CXCR1 results in induced expression of CXCR1 in the NK cells.

The expression levels of CXCR1 vary among different types of NK cells. In some examples, the unmodified primary NK cells isolated from a subject do not express CXCR1. For such NK cells, genetic modification is carried out to express CXCR1. In some other examples, the unmodified primary NK cells isolated from a subject express CXCR1 upon isolation. However, after NK cell expansion, which is necessary for providing sufficient NK cells for clinical uses, the expression of CXCR1 is diminished. For such NK cells, genetic modification is carried out after NK cell expansion to restore the expression of CXCR1 to at least the same level as the level of expression of CXCR1 in the isolated NK cells before cell expansion, or to overexpress CXCR1 to a level higher than the level of expression of CXCR1 in the isolated NK cells before cell expansion. In some other examples, the unmodified primary NK cells isolated from a subject express CXCR1, and the level of expression of CXCR1 does not diminish after NK cell expansion. For such NK cells, genetic modification is carried out to overexpress CXCR1 to a level higher than the level of expression of CXCR1 in the isolated NK cells before or after cell expansion. In some examples, overexpression of CXCR1 results in at least about 10%, or at least about 20%, or at least about 30%, or at least about 40%, or at least about 50%, or at least about 60%, or at least about 70%, or at least about 80%, or at least about 90%, or at least about a 1, 2, 3, 4, 5, 6, 7, 8, 9, 10, 15, 20, 25, 30, 35, 40, 45, 50, 55, 60, 65, 70, 75, 80, 85, 90, 95 or 100 fold increase in expression of CXCR1 in the genetically modified NK cells as compared to the control NK cells.

In some example, expression or overexpression of CXCR1 in the genetically modified NK cells can be transient or stable.

In some examples, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. In one particular example, human peripheral blood mononuclear cells are used to derive NK cells. NK cells can be isolated and expanded by methods well known in the art. In one exemplary method, PBMCs are isolated by gradient centrifugation from buffy coats. After PBMC isolation, cells are co-cultured with γ-irradiated K562 cells in SCGM media (Cellgenix GmbH, Freiburg, Germany) supplemented with FBS and IL2 (Peprotech, Rocky Hills, N.J.). Half media was refreshed every 2-3 days. After 7 days of cell expansion, NK cells are re-stimulated with K562 cells. After 2 rounds of stimulation in total, NK cells can be used for downstream experiments.

In another example, there is provided a method of preparing a natural killer (NK) cell genetically modified to comprise a recombinant nucleic acid encoding C-X-C Motif Chemokine Receptor 1 (CXCR1), the method comprises: (1) obtaining or providing NK cells; (2) providing a recombinant nucleic acid encoding CXCR1; and (3) transferring the recombinant nucleic acid encoding CXCR1 into the NK cell. In some example, some, or the majority, or all of the NK cells into which the recombinant nucleic acid is to be transferred in step (3) are not expressing CXCR1 or have lost the expression of CXCR1. In some other examples, the method further comprises, after step (1), culturing the NK cells to expand the number of NK cells. NK cell expansion methods are known in the art and exemplified in the Experimental Section of the present application.

A recombinant nucleic acid encoding CXCR1 can be created using methods known in the art. For example, the nucleotide sequence coding for CXCR1 can be inserted into an appropriate expression vector, i.e., a vector which contains the necessary elements for the transcription and translation of the inserted protein-coding sequence. The necessary transcriptional and translational signals can also be supplied by the native CXCR1 gene and/or its flanking regions. A variety of host-vector systems may be utilized to express the protein-coding sequence. These include but are not limited to mammalian cell systems infected with virus (e.g., vaccinia virus, adenovirus, etc.), insect cell systems infected with virus (e.g., baculovirus), microorganisms such as yeast containing yeast vectors, or bacteria transformed with bacteriophage, DNA, plasmid DNA, or cosmid DNA, transgenic plants or transgenic non-human animals. The expression elements of vectors vary in their strengths and specificities. Depending on the host-vector system utilized, any one of a number of suitable transcription and translation elements may be used.

Any of the known methods for the insertion of DNA fragments into a vector may be used to construct expression vectors containing a chimeric gene consisting of appropriate transcriptional/translational control signals and the protein coding sequences. Exemplary methods include in vitro recombinant DNA and synthetic techniques. Expression of a nucleic acid sequence encoding CXCR1 may be regulated by a second nucleic acid sequence so that the CXCR1 peptide is expressed in a host transformed with the recombinant DNA molecule. For example, expression of CXCR1 may be controlled by any promoter/enhancer element known in the art. Promoters that are not native CXCR1 promoters which may be used to control CXCR1 expression include, but are not limited to, prokaryotic promoters such as T7 promoter, T7lac promoter, Sp6 promoter, araBAD promoter, trp promoter, lac promoter, Ptac promoter and pL promoter. In a specific example, the promoter used is T7 promoter having the sequence of 5′-TAATACGACTCACTATAGGG-3′ (SEQ ID NO: 3). The T7 promoter functions to initiate transcription of downstream DNA sequences, for example the CXCR1 encoding sequence.

The expression vector as described above can further include a 3′ untranslated region (3′ UTR) following the DNA encoding CXCR1. The 3′ UTR can contain regulatory regions that influence expression of CXCR1 post-transcriptionally, by influencing, for example, polyadenylation, translation efficiency, localization, and/or stability of the mRNA. 3′-UTR contains both binding sites for regulatory proteins as well as microRNAs (miRNAs). By binding to specific sites within the 3′-UTR, miRNAs can decrease gene expression of various mRNAs by either inhibiting translation or directly causing degradation of the transcript. 3′-UTR can also have silencer regions which bind to repressor proteins and inhibit the expression of the mRNA. Many 3′-UTRs also contain AU-rich elements (AREs). Proteins bind AREs to affect the stability or decay rate of transcripts in a localized manner or affect translation initiation. Furthermore, 3′-UTRs can contain the sequence AAUAAA that directs addition of several hundred adenine residues called the poly(A) tail to the end of the mRNA transcript. Poly(A) binding protein (PABP) binds to this tail, contributing to regulation of mRNA translation, stability, and export. For example, poly(A) tail bound PABP interacts with proteins associated with the 5′ end of the transcript, causing a circularization of the mRNA that promotes translation. The 3′-UTR can also contain sequences that attract proteins to associate the mRNA with the cytoskeleton, transport it to or from the cell nucleus, or perform other types of localization. In addition to sequences within the 3′-UTR, the physical characteristics of the region, including its length and secondary structure, contribute to translation regulation. In one example, the 3′UTR used in the CXCR1 expression vector is the 3′UTR from an alpha globin, a beta globin gene, a growth hormone gene. In one specific example, the 3′UTR used is a mouse alpha globin 3′UTR having the sequence of 5′-GCTGCCTTCTGCGGGGCTTGCCTTCTGGCCATGCCCTTCTTCTCTCCCTTGCACCT GTACCTCTTGGTCTTTGAATAAAGCCTGAGTAGGAAG-3′ (SEQ ID NO: 4) (NCBI Reference Sequence NM_008218.2), wherein the underlined sequence is a polyA signal enhancing the stability of mRNA.

In one specific example, the recombinant construct containing the T7 promoter, the nucleotide sequence encoding CXCR1, and the alpha globin 3′UTR, has the following nucleotide sequence: 5′-TAATACGACTCACTATAGGGAAATAAGAGAGAAAAGAAGAGTAAGAAGAAATA TAAGAACCGGTGCCACCATGTCAAATATTACAGATCCACAGATGTGGGATTTTGA TGATCTAAATTTCACTGGCATGCCACCTGCAGATGAAGATTACAGCCCCTGTATG CTAGAAACTGAGACACTCAACAAGTATGTTGTGATCATCGCCTATGCCCTAGTGT TCCTGCTGAGCCTGCTGGGAAACTCCCTGGTGATGCTGGTCATCTTATACAGCAG GGTCGGCCGCTCCGTCACTGATGTCTACCTGCTGAACCTGGCCTTGGCCGACCTA CTCTTTGCCCTGACCTTGCCCATCTGGGCCGCCTCCAAGGTGAATGGCTGGATTTT TGGCACATTCCTGTGCAAGGTGGTCTCACTCCTGAAGGAAGTCAACTTCTACAGT GGCATCCTGCTGTTGGCCTGCATCAGTGTGGACCGTTACCTGGCCATTGTCCATG CCACACGCACACTGACCCAGAAGCGTCACTTGGTCAAGTTTGTTTGTCTTGGCTG CTGGGGACTGTCTATGAATCTGTCCCTGCCCTTCTTCCTTTTCCGCCAGGCTTACC ATCCAAACAATTCCAGTCCAGTTTGCTATGAGGTCCTGGGAAATGACACAGCAA AATGGCGGATGGTGTTGCGGATCCTGCCTCACACCTTTGGCTTCATCGTGCCGCT GTTTGTCATGCTGTTCTGCTATGGATTCACCCTGCGTACACTGTTTAAGGCCCACA TGGGGCAGAAGCACCGAGCCATGAGGGTCATCTTTGCTGTCGTCCTCATCTTCCT GCTTTGCTGGCTGCCCTACAACCTGGTCCTGCTGGCAGACACCCTCATGAGGACC CAGGTGATCCAGGAGAGCTGTGAGCGCCGCAACAACATCGGCCGGGCCCTGGAT GCCACTGAGATTCTGGGATTTCTCCATAGCTGCCTCAACCCCATCATCTACGCCTT CATCGGCCAAAATTTTCGCCATGGATTCCTCAAGATCCTGGCTATGCATGGCCTG GTCAGCAAGGAGTTCTTGGCACGTCATCGTGTTACCTCCTACACTTCTTCGTCTGT CAATGTCTCTTCCAACCTCTGAGTCGACCCAAGCTTGCTGCCTTCTGCGGGGCTT GCCTTCTGGCCATGCCCTTCTTCTCTCCCTTGCACCTGTACCTCTTGGTCTTTGAAT AAAGCCTGAGTAGGAAG-3′ (SEQ ID NO: 5).

In some examples, the basic backbone of the recombinant expression vector is a commercially available vector into which each of the above elements is inserted. Exemplary expression vectors included but are not limited to pFastbac1, pALTER-Ex1, PALTER-Ex2, pCal-n, pCal-n-EK, pCal-c, pCal-Kc, pcDNA 2.1, pDUAL, pET-3a-c, pET-9a-d, pET-11a-d, pET-12a-c, pET-14b, pET-15b, pET-16b, pET-17b, pET-19b, pET-20b(+), pET-21a-d(+), pET-22b(+), pET-23a-d(+), pET-24a-d(+), pET-25b(+), pET-26b(+), pET-27b(+), pET-28a-c(+), pET-29a-c(+), pET-30a-c(+), pET-31b(+), pET-32a-c(+), pET-33b(+), pET-34b(+), pET-35b(+), pET-36b(+), pET-37b(+), pET-38b(+), pET-39b(+), pET-40b(+), pET-41a-c(+), pET-42a-c(+), pET-43a-c(+), pETBlue-1, pETBlue-2, pETBlue-3, pGEMEX-1, pGEMEX-2, pRSET, pTriEx-1, and pTriEx-2 expression vectors. In one specific example the expression vector is a pFastbac1 expression vector.

The nucleic acid encoding CXCR1 can be cloned into the expression vector using molecular cloning techniques commonly known in the art. In some examples, the primers used for molecular cloning are as follows: forward primer 5′-AATAACCGGTGCCACCATGTCAAATATTACAGATCCACAGATG-3′(containing Age-I site) (SEQ ID NO: 6) and reverse primer 5′-TAAAGTCGACTCAGAGGTTGGAAGAGACATTGA-3′(containing Sal-I site) (SEQ ID NO: 7).

Methods generally known in the field can be used for the transcription of mRNA from the recombinant expression vectors described above. For example, PCR can be performed using the recombinant expression vectors as the DNA template to generate the linear DNA template. In vitro transcription of the linear DNA template generated can then be used to generate the CXCR1 mRNA. Optionally, the linear DNA template is purified before in vitro transcription is carried out.

The recombinant nucleic acid encoding CXCR1 to be transferred into the NK cell can be a DNA encoding the CXCR1, or an mRNA obtained by transcription of the DNA. The transfer can be carried out by electroporation, non-viral chemical transfection, viral transduction using retroviral or lentiviral vectors, in a manner commonly used in the art.

In another example, the natural killer (NK) cell modified as described above is further genetically modified to express a recombinant chimeric antigen receptor (CAR). In some examples, the CAR comprises an intracellular signaling domain, a transmembrane domain, and an extracellular domain comprising an antigen binding region. In some examples, the expression of such recombinant CAR allows the genetically modified NK cells to express the antibody binding domain of the CAR on the cell surface, conferring novel antigen specificity that is MHC independent.

The antigen recognition domain includes a polypeptide that is selective for or targets an antigen, receptor, peptide ligand, or protein ligand of the target; or a polypeptide of the target. The antigen recognition domain may be obtained from any of the wide variety of extracellular domains or secreted proteins associated with ligand binding and/or signal transduction. The antigen recognition domain may include a portion of Ig heavy chain linked with a portion of Ig light chain, constituting a single chain fragment variable (scFv) that binds specifically to a target antigen. The antibody may be monoclonal or polyclonal antibody or may be of any type that binds specifically to the target antigen. In another embodiment, the antigen recognition domain can be a receptor or ligand.

The choice of the antigen binding region depends upon the type and number of ligands that define the surface of a target cell. For example, the antigen binding region may be chosen to recognize a ligand that acts as a cell surface marker on target cells associated with a particular disease state. Examples of cell surface markers that can act as ligands for the antigen binding region in the CARs include those associated with or specific to cancer and/or tumor cells, autoimmune diseases, and viral, bacterial and parasitic infections.

In some examples of CARs, the antigen binding region of the extracellular domain binds to a tumor associated antigen, a tumor specific antigen, or a pathogen-specific antigen. The genetically modified NK cells comprising such CARs can then be redirected to the tumor associated antigen, tumor specific antigen or a pathogen-specific antigen based on the antigen binding specificity. A tumor specific antigen is unique to tumor cells and does not occur on other cells in the body. A tumor associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. Tumor associated antigens can be antigens that are expressed on normal cells during fetal development when the immune system is immature and unable to respond or they may be antigens that are normally present at extremely low levels on normal cells but which are expressed at much higher levels on tumor cells. Non-limiting examples of tumor associated or tumor specific antigens include the following: NKG2D, differentiation antigens such as MART-1/MelanA (MART-1), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations such as BCR-ABL, E2A-PRL, H4-RET, 1GH-IGK, MYL-RAR; and viral antigens such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p15, p16, 43-9F, 5T4, 791Tgp72, alpha-fetoprotem, beta-HCG, BCA225, BTAA, CA 125, CA 15-3\CA 27.29\BCAA, CA 195, CA 242, CA-50, CAM43, CD68\I, CO-029, FGF-5, G250, Ga733V, EpCAM, HTgp-175, M344, MA-50, MG7-Ag, MOV18, NB/70K, NY-CO-1, RCAS1, SDCCAG16, TA-90\Mac-2 binding protein, Acyclophilin C-associated protein, TAAL6, TAG72, TLP, and TPS.

In some examples, the CAR recognizes cell-surface tumor associated antigen or tumor specific antigen independent of human leukocyte antigen (HLA) and employs one or more signaling molecules to activate the genetically modified NK cells for killing, proliferation, and/or cytokine production.

In some examples, the antigen binding region of the extracellular domain of the CARs target an antigen that includes but is not limited to CD19, CD20, CD22, ROR1, Mesothelin, CD33/IL3Ra, c-Met, PSMA, Glycolipid F77, EGFRvIII, GD-2, MY-ESO-1 TCR, MAGE A3 TCR, EpCAM and the like.

In some specific examples, the tumor associated antigen is a ligand of NKG2D, and therefore the extracellular domain of the chimeric antigen receptor comprises NKG2D or an antigen binding fragment thereof. In one example, the antigen binding fragment of NKG2D is or comprises the extracellular domain of NKG2D having the following amino acid sequence (from the N-terminal to the C-terminal): FNQEVQIPLTESYCGPCPKNWICYKNNCYQFFDESKNWYESQASCMSQNASLLKVY SKEDQDLLKLVKSYHWMGLVHIPTNGSWQWEDGSILSPNLLTIIEMQKGDCALYAS SFKGYIENCSTPNTYICMQRTV (SEQ ID NO: 8)(UniProt P26718, amino acid residues 83-216), encoded by the following exemplary nucleotide sequence: 5′-TTCAACCAAGAAGTTCAAATTCCCTTGACCGAAAGTTACTGTGGCCCATGTCCTA AAAACTGGATATGTTACAAAAATAACTGCTACCAATTTTTTGATGAGAGTAAAAA CTGGTATGAGAGCCAGGCTTCTTGTATGTCTCAAAATGCCAGCCTTCTGAAAGTA TACAGCAAAGAGGACCAGGATTTACTTAAACTGGTGAAGTCATATCATTGGATG GGACTAGTACACATTCCAACAAATGGATCTTGGCAGTGGGAAGATGGCTCCATT CTCTCACCCAACCTACTAACAATAATTGAAATGCAGAAGGGAGACTGTGCACTCT ATGCCTCGAGCTTTAAAGGCTATATAGAAAACTGTTCAACTCCAAATACGTACAT CTGTATGCAAAGGACTGTG-3′ (SEQ ID NO: 9). NKG2D ligands are structural homologs of MHC class I molecules. NKG2D ligands are absent or rarely expressed in normal tissues, but are extensively expressed in various malignancies and viral-infected tissues. Examples of human NKG2D ligands include class-I-related chains-related molecules A and B (MICA and MICB) proteins and retinoic acid early transcripts-1 (RAET1), also known as UL-16 binding proteins. Examples of mouse NKG2D ligands include five different RAET1 isoforms (RAET1α, RAET1 β, RAET1 γ, RAET1 δ, and RAET1 ε), three different H60 isoforms (H60a, b, and c), and UL16 binding protein 1 (encoded by MULTI gene). Although NKG2D ligands are structural homologs of MHC class I molecules, they do not present antigen to T cells or bind β2-microglobulin. In some examples, the NKG2D ligands for which the extracellular domain of the chimeric antigen receptor binds to are membrane-bound ligands.

In some specific examples, the tumor associated antigen is EpCAM, and therefore the extracellular domain of the chimeric antigen receptor comprises the single-chain variable fragment (scFv) of a monoclonal EpCAM antibody. In one specific example, the scFv of an monoclonal EpCAM antibody has the following amino acid sequence (from the N-terminal to the C-terminal) DIQMTQSPSSLSASVGDRVTITCRSTKSLLHSNGITYLYWYQQKPGKAPKLLIYQMSN LASGVPSRFSSSGSGTDFTLTISSLQPEDFATYYCAQNLEIPRTFGQGTKVELKRATPS HNSHQVPSAGGPTANSGTSGSEVQLVQSGPGLVQPGGSVRISCAASGYTFTNYGMN WVKQAPGKGLEWMGWINTYTGESTYADSFKGRFTFSLDTSASAAYLQINSLRAEDT AVYYCARFAIKGDYWGQGTLLTVSS (SEQ ID NO: 19), encoded by the following exemplary nucleotide sequence: 5′-GATATCCAGATGACCCAGTCCCCGTCCTCCCTGAGTGCTTCTGTTGGTGACCGTG TTACCATCACCTGCCGTTCCACCAAATCCCTCCTGCACTCCAACGGTATCACCTA CCTTTATTGGTATCAACAGAAACCGGGTAAAGCTCCGAAACTTCTGATCTACCAG ATGTCCAACCTGGCTTCCGGTGTTCCGTCTCGTTTCTCCAGTTCTGGTTCTGGTAC CGACTTCACCCTGACCATCTCTTCTCTGCAGCCGGAAGACTTCGCTACCTACTACT GCGCTCAGAACCTGGAAATCCCGCGTACCTTCGGTCAGGGTACCAAAGTTGAAC TTAAGCGCGCTACCCCGTCTCACAACTCCCACCAGGTTCCATCCGCAGGCGGTCC GACTGCTAACTCTGGAACTAGTGGATCCGAAGTACAGCTGGTTCAGTCCGGCCCG GGTCTTGTTCAACCGGGTGGTTCCGTTCGTATCTCTTGCGCTGCTTCTGGTTACAC GTTCACCAACTACGGCATGAACTGGGTCAAACAGGCTCCGGGTAAAGGCCTGGA ATGGATGGGCTGGATCAACACCTACACCGGTGAATCCACCTACGCTGACTCCTTC AAAGGTCGCTTCACTTTCTCCCTCGACACAAGTGCTAGTGCTGCATACCTCCAAA TCAACTCGCTGCGTGCAGAGGATACAGCAGTCTATTACTGCGCCCGTTTCGCTAT CAAAGGTGACTACTGGGGTCAAGGCACGCTGCTGACCGTTTCCTCG-3′ (SEQ ID NO: 18). EpCAM (CD326) is also referred to as EGP-2, 17-1 A, HEA125, MK-1, GA733-2, EGP34, KSA, TROP-1, ESA, TACSTD1 or KS1/4 and is one of the first identified tumor-associated antigens. EpCAM is a type I membrane protein of 314 amino acids (aa) of which only 26 aa are facing the cytoplasm. EpCAM has been postulated to function as a homophilic cell adhesion molecule that interferes with cadherin-mediated cell-cell contact.

In one specific example, the extracellular domain of the CAR has the following amino acid sequence (from the N-terminal to the C-terminal):

(SEQ ID NO: 8)
FNQEVQIPLTESYCGPCPKNWICYKNNCYQFFDESKNWYFSQASCMSQNA
SLLKVYSKEDQDLLKLVKSYHWMGLVHIPTNGSWQWEDGSILSPNLLTII
EMQKGDCALYASSFKGYIENCSTPNTYICMQRTV.

In another specific example, the extracellular domain of the CAR has the following amino acid sequence (from the N-terminal to the C-terminal):

(SEQ ID NO: 19)
DIQMTQSPSSLSASVGDRVTITCRSTKSLLHSNGITYLYWYQQKPGKAPK
LLIYQMSNLASGVPSRFSSSGSGTDFTLTISSLQPEDFATYYCAQNLEIP
RTFGQGTKVELKRATPSHNSHQVPSAGGPTANSGTSGSEVQLVQSGPGLV
QPGGSVRISCAASGYTFTNYGMNWVKQAPGKGLEWMGWINTYTGESTYAD
SFKGRFTFSLDTSASAAYLQINSLRAEDTAVYYCARFAIKGDYWGQGTLL
TVSS

It is understood that the antigen binding region may include some variability within its sequence and still be selective for the targets disclosed herein. Therefore, it is contemplated that the polypeptide of the antigen binding region may be at least 95%, at least 90%, at least 80%, or at least 70% identical to the antigen binding region polypeptide sequences disclosed herein and still be selective for the targets described herein and be within the scope of the disclosure.

The extracellular domain of a chimeric antigen receptor generally also comprises a hinge region. The hinge region is a sequence positioned between for example, the antigen binding region, and the transmembrane domain. The sequence of the hinge region can be obtained from, for example, any suitable sequence from any genus, including human or a part thereof. In some examples, the hinge region includes the hinge region of a human protein including CD-8 alpha, CD28, 4-1BB, OX40, T cell receptor a or 13 chain, a CD3 chain, CD28, CD3c, CD45, CD4, CD5, CD8, CD8a, CD9, CD16, CD22, CD33, CD37, CD64, CD80, CD86, CD134, CD137, ICOS, CD154, functional derivatives thereof, and combinations thereof. In one specific example, the hinge region includes the CD8 hinge region. In some examples, the hinge region can be one selected from, but is not limited to, immunoglobulin (e.g. IgG1, IgG2, IgG3, IgG4, and IgD). In one specific example, the hinge region of the CAR has the following amino acid sequence (from the N-terminal to the C-terminal): FVPVFLPAKPTTTPAPRPPTPAPTIASQPLSLRPEACRPAAG (SEQ ID NO: 14), encoded by the following exemplary nucleotide sequence: 5′-TTCGTGCCGGTCTTCCTGCCAGCGAAGCCCACCACGACGCCAGCGCCGCGACCAC CAACACCGGCGCCCACCATCGCGTCGCAGCCCCTGTCCCTGCGCCCAGAGGCGT GCCGGCCAGCGGCGGGG-3′(SEQ ID NO: 13)

The transmembrane domain of a CAR includes a hydrophobic polypeptide that spans the cellular membrane. In particular, the transmembrane domain spans from one side of a cell membrane (extracellular) through to the other side of the cell membrane (intracellular or cytoplasmic).

In some examples, the transmembrane domain is artificially designed so that more than 25%, more than 50% or more than 75% of the amino acid residues of the domain are hydrophobic residues such as leucine and valine.

The transmembrane domain may be in the form of an alpha helix or a beta barrel, or combinations thereof. The transmembrane domain may include a polytopic protein, which has many transmembrane segments, each alpha-helical, beta sheets, or combinations thereof.

In one example, the transmembrane domain that is naturally associated with one of the domains in the CAR is used. In another example, the transmembrane domain is selected or modified by amino acid substitution to avoid binding of such domains to the transmembrane domains of the same or different surface membrane proteins to minimize interactions with other members of the receptor complex.

For example, a transmembrane domain includes a transmembrane domain of a T-cell receptor a or 13 chain, a CD3 chain, CD28, CD3E, CD45, CD4, CD5, CD7, CD8, CD9, CD16, CD22, CD33, CD37, CD64, CD80, CD86, CD68, CD134, CD137, ICOS, CD41, CD154, functional derivatives thereof, and combinations thereof. In one specific example, the transmembrane domain is a CD8 transmembrane domain. In one specific example, the transmembrane domain of the CAR has the following amino acid sequence (from the N-terminal to the C-terminal): GAVHTRGLDFACDIYIWAPLAGTCGVLLLSLVITLYCNHRN (SEQ ID NO: 10), encoded by the following exemplary nucleotide sequence: 5′-GGCGCAGTGCACACGAGGGGGCTGGACTTCGCCTGTGATATCTACATCTGGGCG CCCTTGGCCGGGACTTGTGGGGTCCTTCTCCTGTCACTGGTTATCACCCTTTACTG CAACCACAGGAAC-3′ (SEQ ID NO: 15).

The intracellular signaling domain of a chimeric antigen receptor is responsible for activation of at least one of the normal effector functions of the immune cell in which the chimeric antigen receptor has been placed. The term “effector function” refers to a specialized function of a differentiated cell, such as a NK cell. The intracellular receptor signaling domain generally includes at least one immunoreceptor tyrosine based activation motif (ITAM)-containing domain. Each ITAM possesses two repeats of the consensus sequence Tyr-X-X-Leu/Ile (X being any amino acid) spaced by six to eight amino acids. The tyrosine residues within ITAM become phosphorylated following interaction of the receptor molecules with their ligands and form docking sites for other proteins involved in the signaling pathways of the cell. In some examples, intracellular receptor signaling domains in the CAR include part or all of CD3, OX40/CD134, FcεRIγ, ICOS/CD278, ILRB/CD122, IL-2RG/CD132, DAP molecules, CD70, cytokine receptor, CD40, or a combination thereof. In one specific example, the intracellular receptor signaling domain used is CD3. In one specific example, the intracellular receptor signaling domain of the CAR has the following amino acid sequence (from the N-terminal to the C-terminal): RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO: 11), encoded by the following exemplary nucleotide sequence: 5′-AGAGTGAAGTTCAGCAGGAGCGCAGACGCCCCCGCGTACCAGCAGGGCCAGAA CCAGCTCTATAACGAGCTCAATCTAGGACGAAGAGAGGAGTACGATGTTTTGGA CAAGAGACGTGGCCGGGACCCTGAGATGGGGGGAAAGCCGAGAAGGAAGAACC CTCAGGAAGGCCTGTACAATGAACTGCAGAAAGATAAGATGGCGGAGGCCTACA GTGAGATTGGGATGAAAGGCGAGCGCCGGAGGGGCAAGGGGCACGATGGCCTT TACCAGGGTCTCAGTACAGCCACCAAGGACACCTACGACGCCCTTCACATGCAG GCCCTGCCCCCTCGC-3′(SEQ ID NO: 16).

In some examples, the chimeric antigen receptors disclosed herein further includes one or more co-stimulatory domains. The inclusion of a co-stimulator can enhance the proliferation, survival and/or development of the NK cells. In some examples, the co-stimulatory domain is a functional signaling domain from a protein including OX40; CD27; CD28; CD30; CD40; PD-1; CD2; CD7; CD258; Natural killer Group 2 member C (NKG2C); Natural killer Group 2 member D (NKG2D), B7-H3; a ligand that binds to at least one of CD83, ICAM-1, LFA-1 (CD11a/CD18), ICOS, and 4-1BB (CD137); CDS; ICAM-1; LFA-1 (CD1a/CD18); CD40; CD27; CD7; B7-H3; NKG2C; PD-1; ICOS; active fragments thereof functional derivatives thereof and combinations thereof.

As used herein, the at least one co-stimulatory domain and intracellular signaling domain may be collectively referred to as the intracellular domain of the chimeric antigen receptor. As used herein, the hinge region and the antigen binding region may be collectively referred to as the extracellular domain of the chimeric antigen receptor.

In some examples, between the extracellular domain and the transmembrane domain of the CAR, or between the intracellular domain and the transmembrane domain of the CAR, there is incorporated a spacer domain. As used herein, the term “spacer domain” generally means any oligo- or polypeptide that functions to link the transmembrane domain to, either the extracellular domain or, the intracellular domain in the polypeptide chain, A spacer domain can comprise up to about 300 amino acids, or about 10 to about 100 amino acids, or about 25 to about 50 amino acids.

In one specific example, the desired CAR in the genetically modified NK cell comprises NKG2D, CD8 alpha hinge and transmembrane domain, and CD3 signaling domains. In one specific example, the recombinant construct containing a T7 promoter, the nucleotide sequence encoding such CAR, and the alpha globin 3′UTR, has the following codon optimized nucleotide sequence: 5′-TAATACGACTCACTATAGGGAAATAAGAGAGAAAAGAAGAGTAAGAAGAAATA TAAGAGCCACCATGCTTCTCCTGGTGACAAGCCTTCTGCTCTGTGAGTTACCACA CCCAGCATTCCTCCTGATCCCAGGCGCGCATGCCTTCAACCAAGAAGTTCAAATT CCCTTGACCGAAAGTTACTGTGGCCCATGTCCTAAAAACTGGATATGTTACAAAA ATAACTGCTACCAATTTTTTGATGAGAGTAAAAACTGGTATGAGAGCCAGGCTTC TTGTATGTCTCAAAATGCCAGCCTTCTGAAAGTATACAGCAAAGAGGACCAGGA TTTACTTAAACTGGTGAAGTCATATCATTGGATGGGACTAGTACACATTCCAACA AATGGATCTTGGCAGTGGGAAGATGGCTCCATTCTCTCACCCAACCTACTAACAA TAATTGAAATGCAGAAGGGAGACTGTGCACTCTATGCCTCGAGCTTTAAAGGCT ATATAGAAAACTGTTCAACTCCAAATACGTACATCTGTATGCAAAGGACTGTGGC TAGCTTCGTGCCGGTCTTCCTGCCAGCGAAGCCCACCACGACGCCAGCGCCGCGA CCACCAACACCGGCGCCCACCATCGCGTCGCAGCCCCTGTCCCTGCGCCCAGAG GCGTGCCGGCCAGCGGCGGGGGGCGCAGTGCACACGAGGGGGCTGGACTTCGCC TGTGATATCTACATCTGGGCGCCCTTGGCCGGGACTTGTGGGGTCCTTCTCCTGTC ACTGGTTATCACCCTTTACTGCAACCACAGGAACAGAGTGAAGTTCAGCAGGAG CGCAGACGCCCCCGCGTACCAGCAGGGCCAGAACCAGCTCTATAACGAGCTCAA TCTAGGACGAAGAGAGGAGTACGATGTTTTGGACAAGAGACGTGGCCGGGACCC TGAGATGGGGGGAAAGCCGAGAAGGAAGAACCCTCAGGAAGGCCTGTACAATG AACTGCAGAAAGATAAGATGGCGGAGGCCTACAGTGAGATTGGGATGAAAGGC GAGCGCCGGAGGGGCAAGGGGCACGATGGCCTTTACCAGGGTCTCAGTACAGCC ACCAAGGACACCTACGACGCCCTTCACATGCAGGCCCTGCCCCCTCGCTGAACCG GTCGACCCAAGCTTGCTGCCTTCTGCGGGGCTTGCCTTCTGGCCATGCCCTTCTTC TCTCCCTTGCACCTGTACCTCTTGGTCTTTGAATAAAGCCTGAGTAGGAAG-3′ (SEQ ID NO: 12).

In another specific example, the desired CAR in the genetically modified NK cell comprises a scFv fragment of an anti-EPCAM antibody, CD8 alpha hinge and transmembrane domain, and CD3 signaling domains. In one specific example, the recombinant construct containing a T7 promoter, the nucleotide sequence encoding such CAR, and the alpha globin 3′UTR, has the following nucleotide sequence: 5′-TAATACGACTCACTATAGGGAAATAAGAGAGAAAAGAAGAGTAAGAAGAAATA TAAGAGCCACCATGCTTCTCCTGGTGACAAGCCTTCTGCTCTGTGAGTTACCACA CCCAGCATTCCTCCTGATCCCAGGCGCGCATGCCTTCAACCAAGAAGTTCAAATT CCCTTGACCGAAAGTTACTGTGGCCCATGTCCTAAAAACTGGATATGTTACAAAA ATAACTGCTACCAATTTTTTGATGAGAGTAAAAACTGGTATGAGAGCCAGGCTTC TTGTATGTCTCAAAATGCCGATATCCAGATGACCCAGTCCCCGTCCTCCCTGAGT GCTTCTGTTGGTGACCGTGTTACCATCACCTGCCGTTCCACCAAATCCCTCCTGCA CTCCAACGGTATCACCTACCTTTATTGGTATCAACAGAAACCGGGTAAAGCTCCG AAACTTCTGATCTACCAGATGTCCAACCTGGCTTCCGGTGTTCCGTCTCGTTTCTC CAGTTCTGGTTCTGGTACCGACTTCACCCTGACCATCTCTTCTCTGCAGCCGGAA GACTTCGCTACCTACTACTGCGCTCAGAACCTGGAAATCCCGCGTACCTTCGGTC AGGGTACCAAAGTTGAACTTAAGCGCGCTACCCCGTCTCACAACTCCCACCAGGT TCCATCCGCAGGCGGTCCGACTGCTAACTCTGGAACTAGTGGATCCGAAGTACA GCTGGTTCAGTCCGGCCCGGGTCTTGTTCAACCGGGTGGTTCCGTTCGTATCTCTT GCGCTGCTTCTGGTTACACGTTCACCAACTACGGCATGAACTGGGTCAAACAGGC TCCGGGTAAAGGCCTGGAATGGATGGGCTGGATCAACACCTACACCGGTGAATC CACCTACGCTGACTCCTTCAAAGGTCGCTTCACTTTCTCCCTCGACACAAGTGCT AGTGCTGCATACCTCCAAATCAACTCGCTGCGTGCAGAGGATACAGCAGTCTATT ACTGCGCCCGTTTCGCTATCAAAGGTGACTACTGGGGTCAAGGCACGCTGCTGAC CGTTTCCTCGGCTAGCTTCGTGCCGGTCTTCCTGCCAGCGAAGCCCACCACGACG CCAGCGCCGCGACCACCAACACCGGCGCCCACCATCGCGTCGCAGCCCCTGTCC CTGCGCCCAGAGGCGTGCCGGCCAGCGGCGGGGGGCGCAGTGCACACGAGGGG GCTGGACTTCGCCTGTGATATCTACATCTGGGCGCCCTTGGCCGGGACTTGTGGG GTCCTTCTCCTGTCACTGGTTATCACCCTTTACTGCAACCACAGGAACAGAGTGA AGTTCAGCAGGAGCGCAGACGCCCCCGCGTACCAGCAGGGCCAGAACCAGCTCT ATAACGAGCTCAATCTAGGACGAAGAGAGGAGTACGATGTTTTGGACAAGAGAC GTGGCCGGGACCCTGAGATGGGGGGAAAGCCGAGAAGGAAGAACCCTCAGGAA GGCCTGTACAATGAACTGCAGAAAGATAAGATGGCGGAGGCCTACAGTGAGATT GGGATGAAAGGCGAGCGCCGGAGGGGCAAGGGGCACGATGGCCTTTACCAGGG TCTCAGTACAGCCACCAAGGACACCTACGACGCCCTTCACATGCAGGCCCTGCCC CCTCGCTGAACCGGTCGACCCAAGCTTGCTGCCTTCTGCGGGGCTTGCCTTCTGG CCATGCCCTTCTTCTCTCCCTTGCACCTGTACCTCTTGGTCTTTGAATAAAGCCTG AGTAGGAAG-3′ (SEQ ID NO: 20)

The terms “CD3ζ”, “CD3zeta”, “CD3z” are used interchangeably herein to refer to the same T-cell surface glycoprotein CD3 zeta chain.

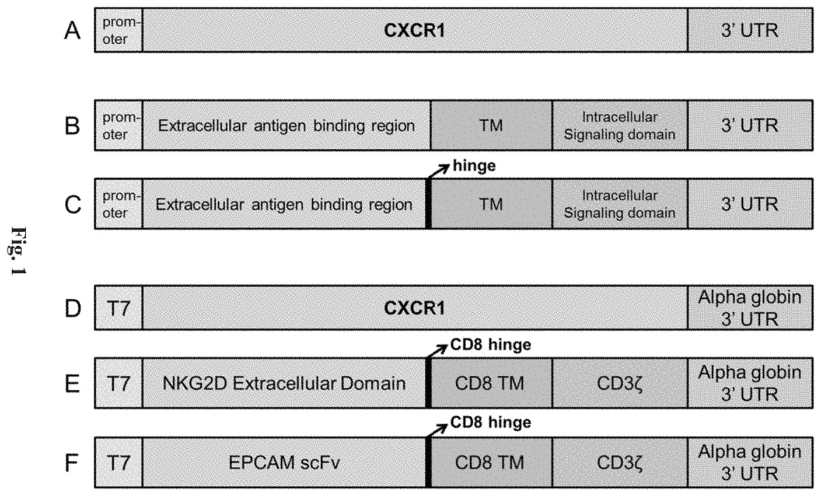

Expression of CARs in the genetically modified NK cells can be transient or stable. In some examples, the constructs in each of FIGS. 1A-1F are transiently or stably expressed in the genetically modified NK cells.

In another example, there is provided a method of preparing a natural killer (NK) cell genetically modified to comprise a recombinant nucleic acid encoding CXCR1, and also expressing a CAR. The method comprises: (1) obtaining or providing NK cells; (2) providing a recombinant nucleic acid encoding CXCR1 and a recombinant nucleic acid encoding the CAR; and (3) transferring the recombinant nucleic acid encoding CXCR1 and the recombinant nucleic acid encoding the CAR into the NK cell. In some examples, some, or the majority, or all of the NK cells into which the recombinant nucleic acids are to be transferred in step (3) are not expressing CXCR1 or have lost the expression of CXCR1. In some other examples, the method further comprises, after step (1), culturing the NK cells to expand the number of NK cells. NK cell expansion methods are known in the art and exemplified in the Experimental Section of the present application. In some examples, the recombinant nucleic acids encoding CXCR1 and the CAR are transferred into the NK cells simultaneously. In some examples wherein the recombinant nucleic acids encoding CXCR1 and the CAR are transferred into the NK cells simultaneously, the recombinant nucleic acids encoding CXCR1 and the CAR are cloned into the same vector. In some other examples, the recombinant nucleic acids encoding CXCR1 and the CAR are transferred into the NK cells sequentially.

The molecular cloning and techniques described above for preparing and transferring the recombinant nucleic acid encoding CXCR1 into the NK cell can also be used for preparing and transferring the recombinant nucleic acid encoding the CAR into the NK cell. In some examples, the recombinant nucleic acid encoding the CAR can be prepared from an amino acid sequence of the specified CAR by any conventional method. A base sequence encoding an amino acid sequence can be obtained from the NCBI RefSeq IDs or accession numbers of GenBenk for an amino acid sequence of each domain, and the nucleic acid as disclosed herein can be prepared using a standard molecular biological and/or chemical procedure. For example, based on the base sequence, a polynucleotide can be synthesized, and the polynucleotide of the present disclosure can be prepared by combining DNA fragments which are obtained from a cDNA library using a polymerase chain reaction (PCR). The following techniques can also be used for introducing the CAR into the NK cell: (i) CARs that are designed to signal through endodomains, (ii) CARs with variable lengths of extracellular domains connecting the antigen-recognition domain to the cell surface, and (iii) artificial antigen presenting cells (aAPC) derived from K562 to expand CAR+ NK cells. In one particular example, the NK cells are genetically modified to express the CAR by electroporation.

In some examples, the modified NK cells are able to replicate in vivo resulting in long-term persistence that can lead to sustained tumor control.

The sequence of the open reading frame encoding the chimeric antigen receptor can be obtained from a genomic DNA source, a cDNA source, or can be synthesized (e.g. via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

In one example, the NK cells as described herein can be provided as a composition or a pharmaceutical composition. The compositions as described herein may be administered in a number of ways depending upon whether local or systemic treatment is desired. Administration may be topical, pulmonary (e.g., by inhalation or insufflation of powders or aerosols, including by nebulizer; intratracheal, intranasal, epidermal and transdermal) or systemic such as oral, and/or parenteral. Parenteral administration includes intravenous, intraarterial, subcutaneous, intraperitoneal or intramuscular injection or infusion; or intracranial, e.g., intrathecal or intraventricular, administration. In some examples, the route of administration may be selected from the group consisting of systemic administration, oral administration, intravenous administration and parenteral administration.

A composition or pharmaceutical composition as described herein can be provided in unit dosage form wherein each dosage unit, e.g., an injection, contains a predetermined amount of the NK cells as disclosed herein, alone or in appropriate combination with other active agents. The term “unit dosage form” as used herein refers to physically discrete units suitable as unitary dosages for human and animal subjects, each unit containing a predetermined quantity of the NK cells as disclosed herein, alone or in combination with other active agents, calculated in an amount sufficient to produce the desired effect, in association with a pharmaceutically acceptable diluent, carrier, or vehicle, where appropriate. The specifications for the unit dosage forms depend on the particular pharmacodynamics associated with the pharmaceutical composition in the particular subject. Unit dosage forms can be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid or semi-liquid carriers.

The compositions as described herein may additionally contain other adjunct components conventionally found in pharmaceutical compositions. Thus, for example, the compositions may contain additional, compatible, pharmaceutically-active materials such as, for example, antipruritic, astringents, local anaesthetics or anti-inflammatory agents, or may contain additional materials useful in physically formulating various dosage forms of the compositions of the present invention, such as buffer, dyes, preservatives, antioxidants, opacifiers, thickening agents and stabilizers or combination thereof appropriate for use with the pharmacologically active agent that may be added to solution in any concentration suitable for use in eye drops. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colourings, flavourings and/or aromatic substances and the like which do not deleteriously interact with the NK cells of the formulation.

In some examples, a medical disease or disorder can be treated by administering a population of the NK cell as disclosed herein. In some examples, the medical disease or disorder is cancer or tumor. Due to their release of pro-inflammatory cytokines, NK cells may reverse the anti-inflammatory tumor microenvironment and increase adaptive immune responses by promoting differentiation, activation, and/or recruitment of accessory immune cell to sites of malignancy.

Thus, in one example, there is provided a method of treating cancer or tumor in a subject in need thereof, comprising administering to the subject a pharmaceutically effective amount of the NK cells or the pharmaceutical composition as disclosed herein. In another example, there is provided use of a pharmaceutically effective amount of the NK cells or the pharmaceutical composition as disclosed herein in the manufacture of a medicament for treating cancer or tumor. In yet another example, there is provided the NK cells or the pharmaceutical composition as disclosed herein for use in treating cancer or tumor. In some examples, the genetically modified NK cells used to treat cancer or tumor are prepared during the course of treatment. Thus, in one example, there is provided a method of treating cancer or tumor in a subject in need thereof, the method comprises: (i) obtaining NK cells from the subject, or from a donor which is different from the subject to be treated; (ii) providing a recombinant nucleic acid encoding CXCR1; (iii) transferring the recombinant nucleic acid encoding CXCR1 into the NK cell to obtain genetically modified NK cells; and (iv) administering to the subject a pharmaceutically effective amount of the NK cells obtained from (iii). In another example, there is provided a method of treating cancer or tumor in a subject in need thereof, the method comprises: (i) obtaining NK cells from the subject, or from a donor which is different from the subject to be treated; (ii) providing a recombinant nucleic acid encoding CXCR1 and a recombinant nucleic acid encoding the CAR; (iii) transferring the recombinant nucleic acid encoding CXCR1 and the recombinant nucleic acid encoding the CAR into the NK cell to obtain genetically modified NK cells; and (iv) administering to the subject a pharmaceutically effective amount of the NK cells obtained from (iii). In some example, some, or the majority, or all of the NK cells into which the recombinant nucleic acid is to be transferred in step (iii) are not expressing CXCR1 or have lost the expression of CXCR1. In some other examples, the method further comprises, after step (i), culturing the NK cells to expand the number of NK cells. NK cell expansion methods are known in the art and exemplified in the Experimental Section of the present application.

The term “pharmaceutically effective amount” as used herein includes within its meaning a sufficient but non-toxic amount of the NK cells as described herein to provide the desired treatment effect. Desirably an effective amount or sufficient number of the NK cells as described herein is present in the composition and introduced into the subject such that long-term, specific, anti-tumor responses are established to reduce the size of a tumor or eliminate tumor growth or regrowth than would otherwise result in the absence of such treatment. Desirably, the amount of NK cells introduced into the subject causes at least a 10%, 20%, 30%, 40%, 50%, 60%, 70%, 80%, 90%, 95%, 98%, or 100% decrease in tumor size when compared to otherwise same conditions wherein the NK cells are not present. The exact amount of NK cells required will vary from subject to subject depending on factors such as the species being treated, the age and general condition of the subject, the severity of the condition being treated (e.g. the stage and/or size of the tumor), the mode of administration, and so forth. In general, the concentration of NK cells desirably should be sufficient to provide in the subject being treated at least from about 1×10⁶ to about 1×10⁹NK cells, even more desirably, from about 1×10⁷ to about 5×10⁸NK cells, although any suitable amount can be utilized either above, e.g., 5×10⁸ cells, or below, e.g., 1×10⁷ cells. The dosing schedule can be based on well-established cell-based therapies, or an alternate continuous infusion strategy can be employed. These values provide general guidance of the range of NK cells to be utilized by the practitioner upon optimizing the method of treatment as disclosed herein. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on inter-individual differences in pharmacokinetics, drug disposition, and metabolism. In any given case, an appropriate “effective amount” may be determined by one of ordinary skill in the art using only routine experimentation.

Cancers or tumors for which the present treatment methods are useful include any malignant cell type, such as those found in a solid tumor or a hematological tumor. In some specific examples, the malignancy is a solid tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. In some specific examples, the malignancy is a hematological tumor. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma. The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia. In some examples, the cancer/tumor that can be treated using the genetically modified NK cells provided herein include colorectal cancer, ovarian cancer, head and neck cancer, liver cancer, breast cancer, cervical cancer, neuroblastoma, osteosarcoma, lymphoma and glioma. In one specific example, the cancer/tumor that can be treated is ovarian cancer. In another specific example, the cancer/tumor than can be treated is head and neck cancer.

In some examples, the cancer/tumor expresses NKG2D ligand or EpCAM. In some other examples, the cancer/tumor expresses NKG2D ligand or EpCAM after treatment with another drug, radiation or biological agent.

The terms “treat,” “treatment,” and grammatical variants thereof, refer to both therapeutic treatment and prophylactic or preventative measures, wherein the object is to prevent or slow down (lessen) an undesired physiological condition, disorder or disease or obtain beneficial or desired clinical results. Such beneficial or desired clinical results include, but are not limited to, alleviation of symptoms; diminishment of extent of condition, disorder or disease; stabilized (i.e. not worsening) state of condition, disorder or disease; delay or slowing of condition, disorder or disease progression; amelioration of the condition, disorder or disease state, remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

The terms “decrease”, “reduced”, “reduction”, “decrease”, “removal” or “inhibit” are all used herein generally to mean a decrease by a statistically significant amount. However, for avoidance of doubt, “reduced”, “reduction” or “decrease”, “removal”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level (e.g., in the absence of a treatment as described herein).

In some examples, the subject or patient to be treated is an animal, mammal, human, including, without limitation, animals classed as bovine, porcine, equine, canine, lupine, feline, murine, ovine, avian, piscine, caprine, corvine, acrine, or delphine. In one example, the patient is a human.

The source of NK cells that can be used for treating a medical disease or disorder may be of any kind, but in specific examples the cells are obtained from a bank of umbilical cord blood, peripheral blood, human embryonic stem cells, or induced pluripotent stem cells.

In some examples, the NK cells that can be used for treating a medical disease or disorder are autologous, i.e. obtained from the same individual to which the treatment is to be administered. For example, autologous source of NK cells can be collected from a patient in need of treatment and NK cells are activated and modified using the methods described herein and known in the art and then infused back into the patient. Some autologous source of NK cells include PBMCs, umbilical cord blood obtained when the patient was born and subsequently preserved, and induced pluripotent stem cells derived from cells obtained from the patient.

In some other examples, the NK cells that can be used for treating a medical disease or disorder are allogeneic, i.e. derived from a different individual of the same species as the patient, such as an NK cell donor. In some other examples, the NK cells that can be used for treating a medical disease or disorder are xenogeneic, i.e. derived from an animal of a different species as the patient. Genetically modified NK cells derived from allogeneic xenogeneic sources can provide an off-the-shelf product.

Allogeneic or autologous NK cells induce a rapid immune response but disappear relatively rapidly from the circulation due to their limited lifespan. Thus, there is reduced concern of persisting side effects using the treatment methods as disclosed herein.

In certain examples, the NK cells as described herein are administered in combination with a second therapeutic agent. For example, the second therapeutic agent may comprise T cells, an immunomodulatory agent, a monoclonal antibody, or a chemotherapeutic agent. In non-limiting examples, the immunomodulatory agent is lenolidomide, the monoclonal antibody is alemtuzumab, rituxumab, trastuzumab, ibritumomab, gemtuzumab, brentuximab, adotranstuzumab, blinatunomab, daratumumab or elotuzumab, and the chemotherapeutic agent is fludarabine or cyclophosphamide.

Following administration of the genetically modified NK cells as disclosed herein for treating or preventing a cancer/tumor, the efficacy of the treatment can be assessed in various ways well known to the skilled practitioner. For instance, one of ordinary skill in the art will understand that a therapeutic genetically modified cell delivered in conjunction with the chemo-adjuvant is efficacious in treating or inhibiting a cancer in a patient by observing that the therapeutic genetically modified cell reduces the cancer cell load or prevents a further increase in cancer cell load. Cancer cell loads can be measured by methods that are known in the art, for example, using polymerase chain reaction assays to detect the presence of certain cancer cell nucleic acids or identification of certain cancer cell markers in the blood using, for example, an antibody assay to detect the presence of the markers in a sample (e.g., but not limited to, blood) from a subject or patient, or by measuring the level of circulating cancer cell antibody levels in the patient.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used herein, the term “about”, in the context of amounts or concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Methods

Tumor Cell Lines

K562 feeder cells engineered to express different cytokines (not disclosed) were maintained in IMDM medium. Ovarian cancer cell lines: SKOV3-luc, SW626 and CaOV3 were maintained in McCoy's 5A medium. All types of medium were supplemented with 10% FBS (Gibco).

Ex Vivo Expansion of Natural Killer Cells

Peripheral blood mononuclear cells (PBMCs) of healthy donors were isolated by gradient centrifugation from buffy coats obtained from Health Sciences Authority (HSA, Singapore) with approval by the university IRB. After PBMC isolation, cells were co-cultured with γ-irradiated K562 cells in SCGM media (Cellgenix GmbH, Freiburg, Germany) supplemented with 10% FBS and 50 IU IL2 (Peprotech, Rocky Hills, N.J.) at a ratio of 1 PBMC:2 K562 cells. Half media was refreshed every 2-3 days. After 7 days of cell expansion, NK cells were re-stimulated with K562 cells at a cell ratio of 1:1. After 2 rounds of stimulation in total, NK cells were used for downstream experiments.

Preparation of CXCR1 and NKG2DCAR DNA Constructs

CXCR1 (SEQ ID NO: 1) was cloned into a pFastbac1 expression vector (Thermo Fisher) modified with a T7 promoter (SEQ ID NO: 3) and an alpha-globin 3′ UTR sequence (SEQ ID NO: 4) using primers 5′-aataACCGGTgccaccatgtcaaatattacagatccacagatg-3′(containing Age-I site) (SEQ ID NO: 6) and 5′-taaaGTCGACtcagaggttggaagagacattga-3′(containing Sal-I site) (SEQ ID NO: 7). FIG. 1A provides a schematic representation of the CXCR1 expression cassette comprised in the final vector.

To generate the NKG2D CAR construct or EpCAM CAR construct, the sequences was designed by in silico assembly of fragments encoding the NKG2D extracellular domain (DNA sequence of SEQ ID NO: 9, corresponding to amino acid sequence of SEQ ID NO: 8 or UniProt P26718, residues 83-216) or the scFv of anti-EpCAM monoclonal antibody (DNA sequence of SEQ ID NO: 18, corresponding to amino acid sequence of SEQ ID NO: 19), the CD8 alpha hinge (DNA sequence of SEQ ID NO: 13, corresponding to amino acid sequence of SEQ ID: 14 or UniProt P01732, residues 128-169) and transmembrane domain (DNA sequence of SEQ ID NO: 15, corresponding to amino acid sequence of SEQ ID NO: 10 or UniProt P01732, residues 170-210) and the CD3 intracellular ITAM domain (DNA sequence of SEQ ID NO: 16, corresponding to amino acid sequence of SEQ ID NO: 11 or UniProt P20963, residues 52-164), followed by synthesis of the fusion gene (AIT Biotech, Singapore). The above NKG2D CAR sequence (SEQ ID NO: 12) was subcloned into pFastbac1 expression vector as described above using Eco-RI and Sal-I. FIG. 1E provides a schematic representation of the NKG2D CAR expression cassette comprised in the final vector.

RNA Preparation and Electroporation

For in vitro transcription of mRNA, PCR was performed using the pFastbac1 vectors as the DNA template, a forward primer CMV-F (5′-atccgctcgagtagttattaatagtaatcaattacggggtc-3′) (SEQ ID NO: 17), and reverse primer containing a synthesized 150-nt poly (A) tail to generate the linear DNA template. The PCR products were then purified with phenol-chloroform. Capped mRNA was generated through in vitro transcription of the PCR DNA templates using the mMESSAGE mMACHINE T7 ULTRA transcription kit (Thermo Fisher). Control RNA template provided in the kit was also generated. RNA pellets were re-suspended in RNase-free water and frozen at −80° C. for storage. To overexpress CXCR1 in NK cells, expanded NK cells was mixed with CXCR1 mRNA and electroporated in a 2-mm cuvette (Bio-Rad, Hercules, Calif.) using an electroporator. For in vitro studies of overexpressing CXCR1 and NKG2D CAR in NK cells, expanded NK cells was mixed with NKG2D CAR mRNA and CXCR1 mRNA and electroporated. To generate NKG2D CAR cells without CXCR1 (NKG2D CAR+ CXCR1-NK cells), CXCR1 mRNA were replaced with control RNA with amount unchanged. NK cells were rested overnight prior to downstream experiments.

Phenotyping

To phenotype NK cells, the following monoclonal antibodies conjugated to PE or APC were used (Becton Dickinson Biosciences, San Diego, Calif.): CD3, CD56, CXCR1, CXCR2 and NKG2D. Cells were acquired using a FACSCalibur flow cytometers (BD Biosciences) or Acuri C6 (BD Biosciences) and analyzed.

NSG Xenograft Animal Model

Animal experiments were performed according to protocols reviewed and approved by Institutional Animal Care and Use Committee (IACUC), the Biological Resource Centre (BRC), the Agency for Science, Technology and Research (A*STAR), Singapore. Non-obese diabetic/severe combined immuno-deficiency/IL-2Rγcnull (NSG) mice (The Jackson Laboratory) were maintained and used in the current study. All luminescent signals and images were acquired and analyzed with the Xenogen living imaging software v3.2.

In Vivo Migration Assay

To examine improved migration towards IL8-secreting ovarian cancer tumors, female NSG mice (8-10 weeks) were injected i.p with 1×10⁷ SKOV3-luc tumor cells. On day 10 post-tumor inoculation, tumor engraftment was confirmed by live bioluminescence imaging. Two groups of mice were then injected via the tail vein with either 5×10⁶ CXCR1+ NK cells or Mock NK cells labelled with DiR (Perkin Elmer, Ohio, USA). Following NK cell infusion, mice received single dose of 20,000 IU/mouse recombinant human IL-2 intraperitoneally (i.p). Biodistribution of NK cells was examined by DIR imaging at time points of 24 and 48 hours. Peritoneal tumors were isolated after 48 hours and imaged. Photon emission was analyzed by constant region-of-interest (ROI) drawn over the tumor region and the signal measured as radiant efficiency.

In Vivo Therapeutic Efficacy Evaluation

To determine if the CXCR1 overexpression on NK cells can improve therapeutic efficacy towards ovarian peritoneal carcinomas in NSG mice, 8-10 weeks old female NSG mice were used. Mice were injected i.p. with 1×10⁷ SKOV3 luc cells and on day 7 post-tumor inoculation, tumor engraftment was confirmed by live bioluminescence imaging (BLI) monitored using an IVIS100 Imaging platform. Mice with similar BLI signal intensity randomly divided into 3 different treatment groups containing 5 mice per group. On day 7, 100 μl of PBS or 100 μl cell suspension containing 1×10⁷ NK cells of two different group: (1) Mock NK cells, (2) CXCR1+NK cells was injected intravenously (i.v.). A total of four injections at the same dose were given in a 3-4 days interval. To prolong the longevity of NK cells in vivo, mice received dose of 20,000 IU/mouse recombinant human IL-2 intraperitoneally (i.p) every 2-3 days from the start of NK cell inoculation till 4 days after the last NK cell injection. Tumor progression was monitored by BLI every week up to 42 days post tumor inoculation, after which survival of mice was monitored.

To determine if the overexpression of CXCR1 in NK2GD CAR modified NK cells can improve therapeutic efficacy towards ovarian peritoneal carcinomas in NSG mice, the above described experiment was repeated with the following changes: On day 7, 100 μl of PBS or 100 μl cell suspension containing 1×10⁷ NK cells of two different group: (1) NKG2D CAR+ CXCR1− NK cells, (2) NKG2D CAR+ CXCR1+ NK cells was injected intravenously (i.v.). The frequency, dose and route of NK cell and IL-2 injection remain unchanged as the above described experiment. Tumor progression was monitored by BLI every week up to 42 days post tumor inoculation, after which survival of mice was monitored.

Mice were monitored closely and humanely euthanized after observing the development of moribund condition characterized by obvious abdominal bloating due to ascites, palpable hypothermia, inability to walk, ruffled fur, hunched posture and/or lack of overt response to manipulation.

Head and Neck Subcutaneous Animal Model

To establish a head and neck subcutaneous model, 5×10⁶ Fadu tumour cells were subcutaneously injected in the left flank in male NSG mice (8-10 weeks). After tumours were established and palpable, NK cells (5×10⁶) were labelled with Xenolight DiR (Perkin Elmer, Ohio, USA) and injected via the tail vein (intravenous; i.v.) into tumor-bearing mice. Following NK cell infusion, mice received a single dose of 20,000 IU/mouse recombinant human IL-2 intraperitoneally (i.p). Bio-distribution of NK cells was examined by DIR imaging at time points indicated and using the IVIS imaging system (Xenogen). Photon emission was analyzed by constant region-of-interest (ROI) drawn over the tumour region. Subcutaneous tumours were isolated after 72 hours, respectively, and imaged again ex vivo using the IVIS imaging system. Photon emission was analyzed by constant region-of-interest (ROI) drawn over the tumour region and the signal measured as radiant efficiency.

Statistical Analyses

Data are expressed as means±standard deviation. Statistics were computed using GraphPad Prism 7.0 (GraphPad Software). For in vitro and in vivo experiments, unpaired Student's t test was used to evaluate continuous variable of 2 groups, and 1-way ANOVA with post-test Bonferroni to evaluate continuous variables of more than 2 groups. Survival was analyzed by the Kaplan-Meier method and the log-rank (Mantel-Cox) test to compare pairs of groups. Differences were considered significant when the P value was less than 0.05.

Results

Overexpression of CXCR1 Promotes NK Cell Tumor Migration Towards Tumor Cells In Vitro

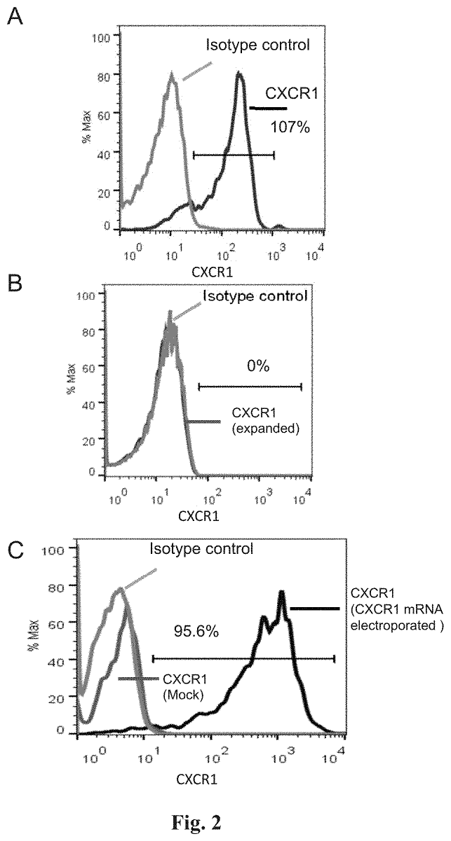

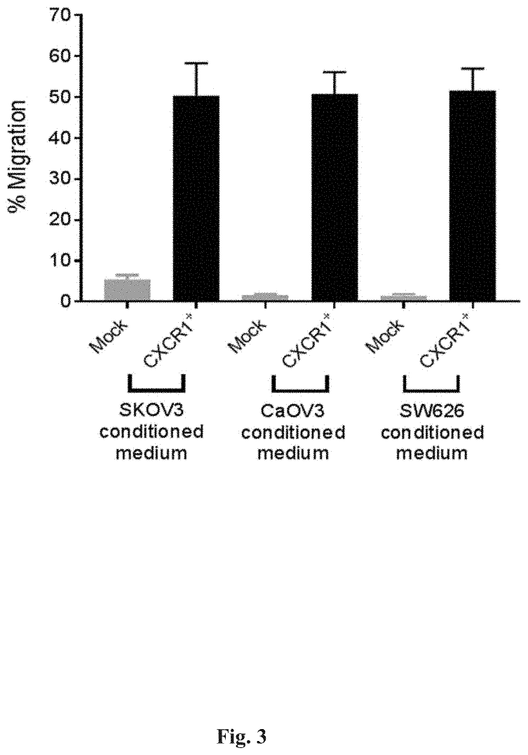

CXCR1 expression in NK cells was investigated. While the majority of freshly isolated NK cells express CXCR1, after NK cell expansion, which is necessary for clinical treatment, CXCR1 expression diminished in the ex vivo expanded primary NK cells (FIG. 2 A, B). Electroporation of NK cells with mRNA encoding CXCR1 successfully restored and elevated CXCR1 expression in the ex vivo expanded NK cells (FIG. 2C). The migration of the transfected NK cells towards three different human cancer cells was then tested. As shown in FIG. 3, the condition media generated by these cancer cells was effective to attract CXCR1 modified NK cells but not those without CXCR1 modification (mock controls), demonstrating that CXCR1 overexpression increases the migration ability of ex vivo expanded NK cells towards cancer cells.

Overexpression of CXCR1 Promotes NK Cell Tumor Migration Towards Tumor Sites In Vivo

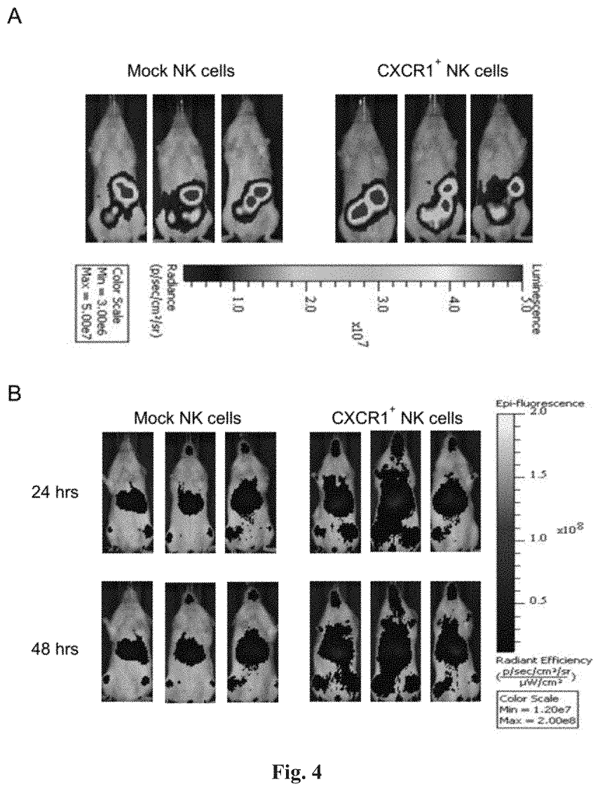

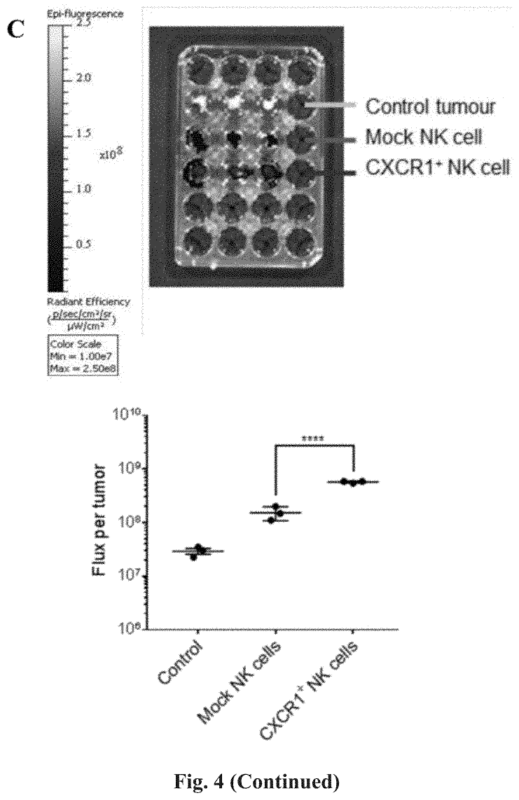

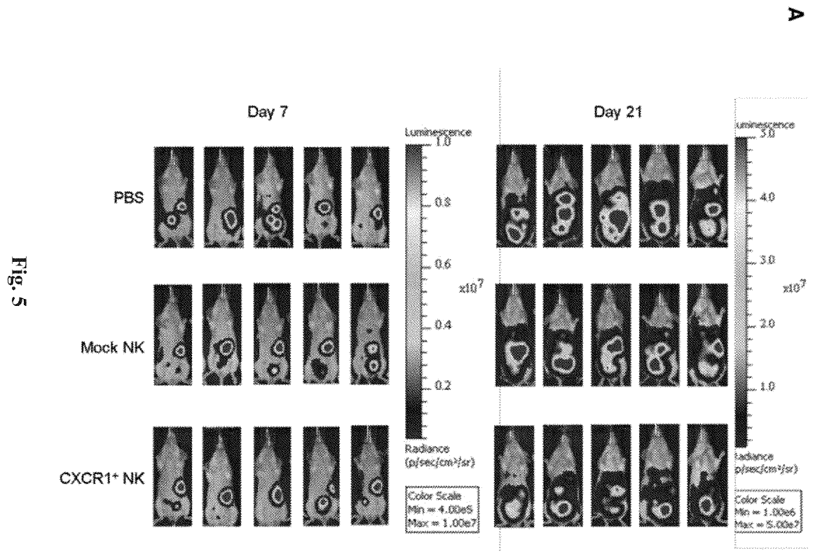

It was then investigated whether the enhanced NK cell migration via overexpression of CXCR1 could be established in vivo. A human SKOV3 ovarian peritoneal carcinoma xenograft model was established in mice by intraperitoneal injection of SKOV3-luc cells, a tumor cell line genetically modified with firefly luciferase gene. Ten days after tumor inoculation, the mice were randomized based on ROI values obtained via BLI imaging and similar tumor burden was used as a criterion for randomization. The mice were then intravenously injected through tail vein with mock NK cells or CXCR1 overexpressing NK cells. The NK cells were pre-labelled with DIR dye, a lipophilic, NIR fluorescent cyanine dye ideal for staining cytoplasmic membrane. FIG. 4A shows bioluminescence imaging results: similar tumor burdens were observed in SKOV3-inoculated NSG mice that received mock NK cells or CXCR1+NK cells, before NK cell injection. FIG. 4B addresses whether there was an increase in DIR signal at the peritoneal region of the mice inoculated with SKOV3 cancer cells. Increased DIR signals over the peritoneal region were observed in the mice receiving CXCR1+NK cells, but not in those injected with mock NK cells. Furthermore, isolated tumors from the peritoneal cavity of sacrificed mice were studied using ex vivo imaging. FIG. 4C showed that the DIR signals of tumor tissues from the mice injected with CXCR1+NK cells were significantly higher compared to those from the mice receiving mock NK cells. The flux value differences were statistically significant between two groups, P<0.0001. Therefore, overexpression of CXCR1 on NK cells can improve the migration of intravenously (i.v.) injected NK cells towards peritoneal tumors.

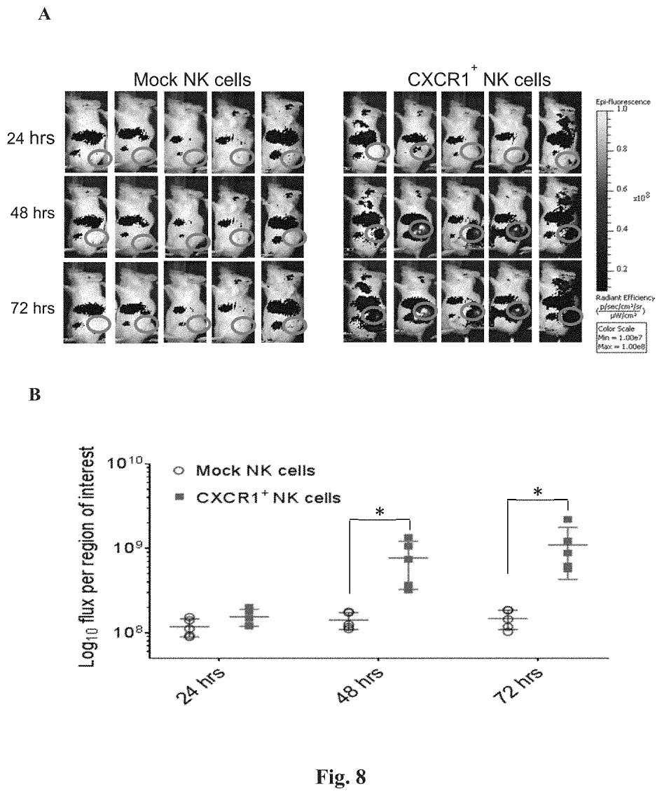

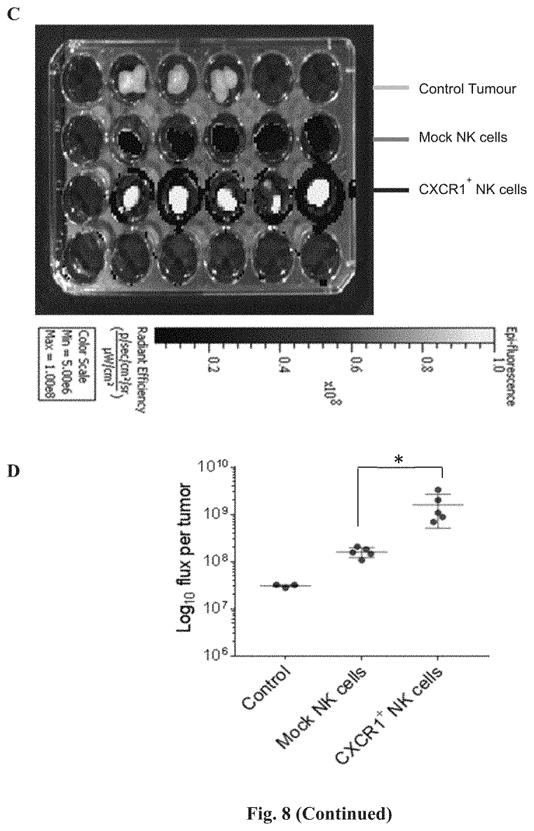

A human head and neck cancer xenograft model was established to investigate whether overexpression of CXCR1 could enhance migration of NK cells toward subcutaneous tumor cells in vivo. The xenograft model was established by inoculating FaDu human hypopharyngeal cancer cells subcutaneously (s.c.) at the left flank of NSG mice. 10 days after tumor inoculation, NK cells were labeled with 1,1′-dioctadecyl-3,3,3′,3′-tetramethylindotricarbocyanine iodide or DiOC18(7) (DiR), a lipophilic, near-infrared (NIR) fluorescent cyanine dye ideal for staining cytoplasmic membrane, and DiR-labeled NK cells were intravenously (i.v.) injected through the tail vein. Cell distribution in mice was imaged every 24 h for 3 days. Whereas DiR fluorescence signals remained in the lung region by 24 h post-NK cell injection, enhanced DiR signals in the tumor sites were observed by 48 h in mice injected with CXCR1 expressing NK cells, which became more pronounced by 72 h (FIG. 8A). Whole-body imaging was unable to detect DiR signals in the tumor sites in mice injected with mock NK cells during the 72-h period. The quantitative analysis of DiR fluorescence signal intensity at the tumor sites demonstrated an approximately 10-fold increase in signal intensity in mice injected with CXCR1 expressing NK cells over those injected with mock NK cells by 48 and 72 h (FIG. 8A). Mice were euthanized after 72 h, and the subcutaneous tumors were collected for ex vivo imaging. The images of the tumors and the associated flux values are shown in FIG. 8B. Similarly, an approximately 10-fold increase in signal intensity was observed in mice injected with CXCR1 expressing NK cells as compared to those injected with mock NK cells. Thus, overexpression of CXCR1 in NK cells can improve the migration/infiltration of i.v.-injected NK cells toward subcutaneous tumors.

Overexpression of CXCR1 Increases the Cancer Killing Capacity of NK Cells In Vivo

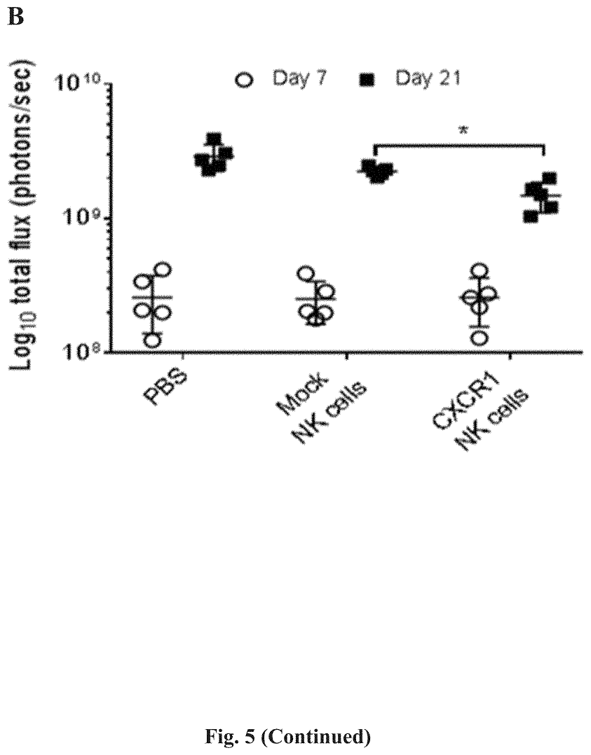

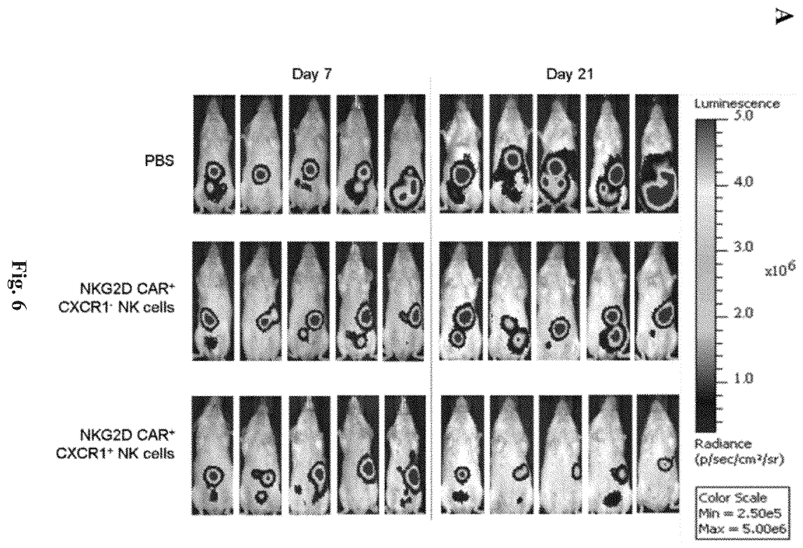

Another mouse experiment was performed to examine whether the improved migration could enhance the in vivo killing capacity of CXCR1 overexpressing NK cells against cancer cells. The same tumor model as described above was used, and the mice inoculated with SKOV3-luc cells were treated with three different ways: PBS, NK cells without CXCR1, and NK cells overexpressing CXCR1. The treatments via intravenous tail vein injection started 7 days after tumor inoculation. As shown in FIG. 5A, tumor burdens were similar among the three groups of mice at day 7. However, tumor growth was slowed in mice treated with NK cells overexpressing CXCR1, as demonstrated by bioluminescence imaging. The flux value differences were analyzed statistically, and the values from mice treated with NK cells overexpressing CXCR1 were significantly lower than those from the two control groups, P<0.05 (FIG. 5B).

Overexpression of CXCR1 Increases Cancer Killing Capacity in NKG2D CAR-NK Cells In Vivo

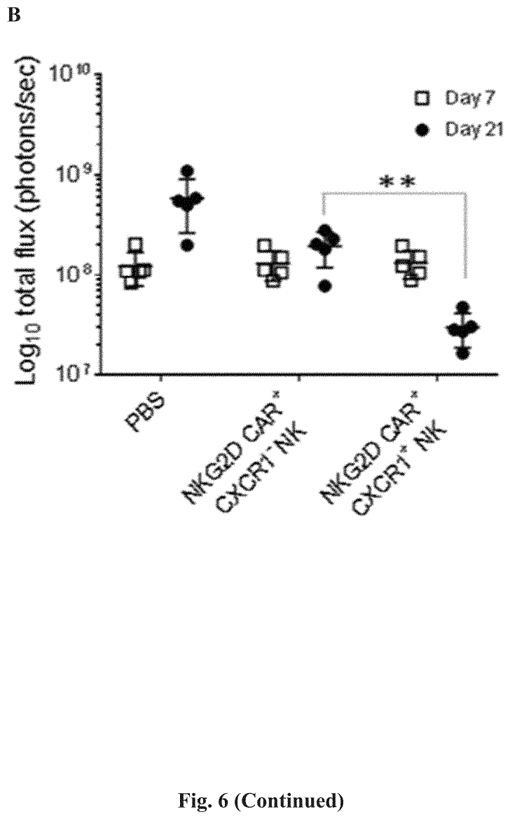

To investigate the effect of combining a chemokine receptor and a CAR on expanded primary NK cells, mRNAs of CXCR1 and a CAR specific to NKG2D ligands was electroporated to the expanded primary NK cells. The SKOV3-luc tumor model was used again, and the mice inoculated with SKOV3-luc cells were treated via intravenous tail vein injection with three different materials: PBS, NK cells expressing NKG2D CAR, and NK cells co-expressing NKG2D CAR and CXCR1. The treatments started 7 days after tumor inoculation. Bioluminescence imaging demonstrated that the tumor burdens in mice receiving NKG2D CAR+ CXCR1+ NK cells was obviously reduced relative to the initial tumor burdens during the course of treatment (FIG. 6A). There was a statistically significant difference in flux values over the peritoneal region (P value <0.01) in the tumor burden between the group with NKG2D CAR+ CXCR1− NK cells and the group with NKG2D CAR+ CXCR1+ NK cells (FIG. 6B). Therefore, NKG2D CAR-modified, CXCR1 overexpressing NK cells display better anti-tumor efficacy than NKG2D CAR-modified NK cells without CXCR1 overexpression. Thus, introducing both CXCR1 and NKG2D CAR can effectively control tumor growth in an ovarian peritoneal carcinoma model.

Overexpression of CXCR1 Increases Cancer Killing Capacity in EpCAM CAR-NK Cells In Vitro

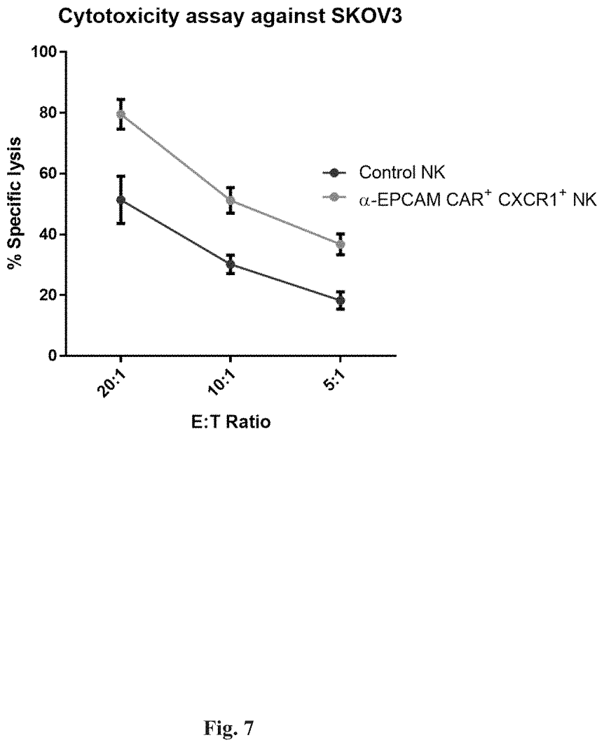

To demonstrate that CXCR1 modified NK cells can also be combined with a conventional CAR construct for enhancing cancer cell killing capacity, expanded primary NK cells were electroporated with mRNAs of CXCR1 and EpCAM CAR that contains a scFv for EpCAM recognition, and tested their in vitro cancer killing capacity against SKOV3, a human ovarian cancer cell line known to express EpCAM. As shown in FIG. 7, the modified NK cells showed an improved in vitro killing capacity against SKOV3 cancer cells over mock-control NK cells, demonstrating that CXCR1 incorporation is compatible with EpCAM CAR, and the potential of CXCR1 modified CAR NK cells to eliminate tumors expressing targets other than NKG2D ligands. Therefore, a conventional CAR with a scFv fragment of a monoclonal antibody (in this case an anti-EpCAM monoclonal antibody) can also be used in CXCR1 overexpressed NK cells for NK cell-mediated anticancer immunotherapy.

1. A natural killer (NK) cell genetically modified to comprise a recombinant nucleic acid encoding C-X-C Motif Chemokine Receptor 1 (CXCR1).

2. The NK cell of claim 1, further genetically modified to express a recombinant chimeric antigen receptor (CAR) comprising an intracellular signaling domain, a transmembrane domain, and an extracellular domain comprising an antigen binding region.

3. The NK cell of claim 2, wherein the intracellular signaling domain comprises at least one immunoreceptor tyrosine based activation motif (ITAM)-containing domain.

4. The NK cell of claim 2, wherein the intracellular signaling domain is CD3ζ.

5. The NK cell of claim 2, wherein the transmembrane domain is CD8 transmembrane domain.

6. The NK cell of claim 2, wherein the antigen binding region binds a tumor associated antigen.

7. The NK cell of claim 6, wherein the tumor associated antigen is solid tumor associated antigen.

8. The NK cell of claim 2, wherein the extracellular domain of the recombinant CAR comprises the extracellular domain of an NK cell activating receptor or a scFv fragment of a monoclonal antibody.

9. The NK cell of claim 2, wherein the extracellular domain of the recombinant CAR comprises the extracellular domain of an NKG2D receptor or a scFv fragment of an anti-EPCAM monoclonal antibody.

10. The NK cell of claim 2, wherein the recombinant CAR further comprises, between the antigen binding region and the transmembrane region, a hinge region.

11. The NK cell of claim 10, wherein the hinge region is a CD8 hinge region.

12. A pharmaceutical composition comprising a pharmaceutically effective amount of the NK cell of claim 1 and a pharmaceutically acceptable excipient.

13. A method of treating cancer or tumor in a subject in need thereof, comprising administering to the subject a pharmaceutically effective amount of the NK cell of claim 1.

14. The method of claim 13, wherein the NK cells are derived from allogeneic or autologous cells.

15. The method of claim 13, wherein the NK cells are derived from peripheral blood, cord blood, bone marrow, or induced pluripotent stem cells.

16. A method of treating cancer or tumor in a subject in need thereof, the method comprises:
(i) obtaining NK cells from the subject, or from a donor which is different from the subject to be treated;
(ii) providing a recombinant nucleic acid encoding CXCR1;
(iii) transferring the recombinant nucleic acid encoding CXCR1 into the NK cell to obtain genetically modified NK cells; and
(iv) administering to the subject a pharmaceutically effective amount of the NK cells obtained from (iii).

17. A method of treating cancer or tumor in a subject in need thereof, the method comprises:
(i) obtaining NK cells from the subject, or from a donor which is different from the subject to be treated;
(ii) providing a recombinant nucleic acid encoding CXCR1 and a recombinant nucleic acid encoding a recombinant chimeric antigen receptor (CAR);
(iii) transferring the recombinant nucleic acid encoding CXCR1 and the recombinant nucleic acid encoding the recombinant CAR into the NK cell to obtain genetically modified NK cells; and
(iv) administering to the subject a pharmaceutically effective amount of the NK cells obtained from (iii).

18. The method of claim 13, wherein cancer or tumor is a solid tumor.

19. A method of preparing the NK cell of claim 1, the method comprises:
(i) obtaining or providing NK cells;
(ii) providing a recombinant nucleic acid encoding CXCR1; and
(iii) transferring the recombinant nucleic acid encoding CXCR1 into the NK cell;
preferably, the recombinant nucleic acid in step (iii) is carried out using electroporation.

20. A method of preparing the NK cell of claim 2, the method comprises:
(i) obtaining or providing NK cells;
(ii) providing a recombinant nucleic acid encoding CXCR1 and a recombinant nucleic acid encoding the recombinant CAR; and
(iii) transferring the recombinant nucleic acid encoding CXCR1 and the recombinant nucleic acid encoding the recombinant CAR into the NK cell;
preferably, the recombinant nucleic acids in step (iii) are carried out using electroporation.

21. (canceled)


SEQUENCE LISTING (20 sequences)

SEQ ID NO: 1 - DNA, 1053 bp, Homo sapiens
atgtcaaatattacagatccacagatgtgggattttgatgatctaaatttcactggcatgccacctgcagatgaagattacagcccctgtatgctagaaactgagacactcaacaagtatgttgtgatcatcgcctatgccctagtgttcctgctgagcctgctgggaaactccctggtgatgctggtcatcttatacagcagggtcggccgctccgtcactgatgtctacctgctgaacctggccttggccgacctactctttgccctgaccttgcccatctgggccgcctccaaggtgaatggctggatttttggcacattcctgtgcaaggtggtctcactcctgaaggaagtcaacttctacagtggcatcctgctgttggcctgcatcagtgtggaccgttacctggccattgtccatgccacacgcacactgacccagaagcgtcacttggtcaagtttgtttgtcttggctgctggggactgtctatgaatctgtccctgcccttcttccttttccgccaggcttaccatccaaacaattccagtccagtttgctatgaggtcctgggaaatgacacagcaaaatggcggatggtgttgcggatcctgcctcacacctttggcttcatcgtgccgctgtttgtcatgctgttctgctatggattcaccctgcgtacactgtttaaggcccacatggggcagaagcaccgagccatgagggtcatctttgctgtcgtcctcatcttcctgctttgctggctgccctacaacctggtcctgctggcagacaccctcatgaggacccaggtgatccaggagagctgtgagcgccgcaacaacatcggccgggccctggatgccactgagattctgggatttctccatagctgcctcaaccccatcatctacgccttcatcggccaaaattttcgccatggattcctcaagatcctggctatgcatggcctggtcagcaaggagttcttggcacgtcatcgtgttacctcctacacttcttcgtctgtcaatgtctcttccaacctctga

SEQ ID NO: 2 - protein, 350 aa, Homo sapiens
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 3 - DNA, 20 bp, Artificial Sequence
  T7 promoter
taatacgactcactataggg

SEQ ID NO: 4 - DNA, 93 bp, Artificial Sequence
  alpha-globin 3′ UTR
gctgccttctgcggggcttgccttctggccatgcccttcttctctcccttgcacctgtacctcttggtctttgaataaagcctgagtaggaag

SEQ ID NO: 5 - DNA, 1230 bp, Artificial Sequence
  CXCR1 expression cassette comprising a T7 promoter, a CXCR1 coding sequence and an alpha globin 3′UTR
taatacgactcactatagggaaataagagagaaaagaagagtaagaagaaatataagaaccggtgccaccatgtcaaatattacagatccacagatgtgggattttgatgatctaaatttcactggcatgccacctgcagatgaagattacagcccctgtatgctagaaactgagacactcaacaagtatgttgtgatcatcgcctatgccctagtgttcctgctgagcctgctgggaaactccctggtgatgctggtcatcttatacagcagggtcggccgctccgtcactgatgtctacctgctgaacctggccttggccgacctactctttgccctgaccttgcccatctgggccgcctccaaggtgaatggctggatttttggcacattcctgtgcaaggtggtctcactcctgaaggaagtcaacttctacagtggcatcctgctgttggcctgcatcagtgtggaccgttacctggccattgtccatgccacacgcacactgacccagaagcgtcacttggtcaagtttgtttgtcttggctgctggggactgtctatgaatctgtccctgcccttcttccttttccgccaggcttaccatccaaacaattccagtccagtttgctatgaggtcctgggaaatgacacagcaaaatggcggatggtgttgcggatcctgcctcacacctttggcttcatcgtgccgctgtttgtcatgctgttctgctatggattcaccctgcgtacactgtttaaggcccacatggggcagaagcaccgagccatgagggtcatctttgctgtcgtcctcatcttcctgctttgctggctgccctacaacctggtcctgctggcagacaccctcatgaggacccaggtgatccaggagagctgtgagcgccgcaacaacatcggccgggccctggatgccactgagattctgggatttctccatagctgcctcaaccccatcatctacgccttcatcggccaaaattttcgccatggattcctcaagatcctggctatgcatggcctggtcagcaaggagttcttggcacgtcatcgtgttacctcctacacttcttcgtctgtcaatgtctcttccaacctctgagtcgacccaagcttgctgccttctgcggggcttgccttctggccatgcccttcttctctcccttgcacctgtacctcttggtctttgaataaagcctgagtaggaag

SEQ ID NO: 6 - DNA, 43 bp, Artificial Sequence
  CXCR1 primer 1
aataaccggtgccaccatgtcaaatattacagatccacagatg

SEQ ID NO: 7 - DNA, 33 bp, Artificial Sequence
  CXCR1 primer 2
taaagtcgactcagaggttggaagagacattga

SEQ ID NO: 8 - protein, 134 aa, Artificial Sequence
  extracellular domain of NKG2D
FNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 9 - DNA, 402 bp, Artificial Sequence
  extracellular domain of NKG2D
ttcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgtatgcaaaggactgtg

SEQ ID NO: 10 - protein, 41 aa, Artificial Sequence
  CD8 transmembrane domain (protein)
GAVHTRGLDFACDIYIWAPLAGTCGVLLLSLVITLYCNHRN

SEQ ID NO: 11 - protein, 112 aa, Artificial Sequence
  CD3z intracellular receptor signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 1249 bp, Artificial Sequence
  NKG2D CAR
taatacgactcactatagggaaataagagagaaaagaagagtaagaagaaatataagagccaccatgcttctcctggtgacaagccttctgctctgtgagttaccacacccagcattcctcctgatcccaggcgcgcatgccttcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgtatgcaaaggactgtggctagcttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaaccggtcgacccaagcttgctgccttctgcggggcttgccttctggccatgcccttcttctctcccttgcacctgtacctcttggtctttgaataaagcctgagtaggaag

SEQ ID NO: 13 - DNA, 126 bp, Artificial Sequence
  CD8 hinge region
ttcgtgccggtcttcctgccagcgaagcccaccacgacgccagcgccgcgaccaccaacaccggcgcccaccatcgcgtcgcagcccctgtccctgcgcccagaggcgtgccggccagcggcgggg

SEQ ID NO: 14 - protein, 42 aa, Artificial Sequence
  CD8 hinge (protein)
FVPVFLPAKPTTTPAPRPPTPAPTIASQPLSLRPEACRPAAG

SEQ ID NO: 15 - DNA, 123 bp, Artificial Sequence
  CD8 transmembrane domain
ggcgcagtgcacacgagggggctggacttcgcctgtgatatctacatctgggcgcccttggccgggacttgtggggtccttctcctgtcactggttatcaccctttactgcaaccacaggaac

SEQ ID NO: 16 - DNA, 336 bp, Artificial Sequence
  CD3z intracellular receptor signaling domain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 17 - DNA, 41 bp, Artificial Sequence
  CMV-F primer
atccgctcgagtagttattaatagtaatcaattacggggtc

SEQ ID NO: 18 - DNA, 762 bp, Artificial Sequence
  EpCAM scFv
gatatccagatgacccagtccccgtcctccctgagtgcttctgttggtgaccgtgttaccatcacctgccgttccaccaaatccctcctgcactccaacggtatcacctacctttattggtatcaacagaaaccgggtaaagctccgaaacttctgatctaccagatgtccaacctggcttccggtgttccgtctcgtttctccagttctggttctggtaccgacttcaccctgaccatctcttctctgcagccggaagacttcgctacctactactgcgctcagaacctggaaatcccgcgtaccttcggtcagggtaccaaagttgaacttaagcgcgctaccccgtctcacaactcccaccaggttccatccgcaggcggtccgactgctaactctggaactagtggatccgaagtacagctggttcagtccggcccgggtcttgttcaaccgggtggttccgttcgtatctcttgcgctgcttctggttacacgttcaccaactacggcatgaactgggtcaaacaggctccgggtaaaggcctggaatggatgggctggatcaacacctacaccggtgaatccacctacgctgactccttcaaaggtcgcttcactttctccctcgacacaagtgctagtgctgcatacctccaaatcaactcgctgcgtgcagaggatacagcagtctattactgcgcccgtttcgctatcaaaggtgactactggggtcaaggcacgctgctgaccgtttcctcg

SEQ ID NO: 19 - protein, 254 aa, Artificial Sequence
  EpCAM scFv (protein)
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRATPSHNSHQVPSAGGPTANSGTSGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 20 - DNA, 1759 bp, Artificial Sequence
  EpCAM CAR
taatacgactcactatagggaaataagagagaaaagaagagtaagaagaaatataagagccaccatgcttctcctggtgacaagccttctgctctgtgagttaccacacccagcattcctcctgatcccaggcgcgcatgccttcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccgatatccagatgacccagtccccgtcctccctgagtgcttctgttggtgaccgtgttaccatcacctgccgttccaccaaatccctcctgcactccaacggtatcacctacctttattggtatcaacagaaaccgggtaaagctccgaaacttctgatctaccagatgtccaacctggcttccggtgttccgtctcgtttctccagttctggttctggtaccgacttcaccctgaccatctcttctctgcagccggaagacttcgctacctactactgcgctcagaacctggaaatcccgcgtaccttcggtcagggtaccaaagttgaacttaagcgcgctaccccgtctcacaactcccaccaggttccatccgcaggcggtccgactgctaactctggaactagtggatccgaagtacagctggttcagtccggcccgggtcttgttcaaccgggtggttccgttcgtatctcttgcgctgcttctggttacacgttcaccaactacggcatgaactgggtcaaacaggctccgggtaaaggcctggaatggatgggctggatcaacacctacaccggtgaatccacctacgctgactccttcaaaggtcgcttcactttctccctcgacacaagtgctagtgctgcatacctccaaatcaactcgctgcgtgcagaggatacagcagtctattactgcgcccgtttcgctatcaaaggtgactactggggtcaaggcacgctgctgaccgtttcctcggctagcttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaaccggtcgacccaagcttgctgccttctgcggggcttgccttctggccatgcccttcttctctcccttgcacctgtacctcttggtctttgaataaagcctgagtaggaag